(19) United States
(12) Patent Application Publication
(10) Pub. No.: US 2009/0321196 A1
(43) Pub. Date: Dec. 31, 2009

(54) Asymmetric Tension Adjustment Mechanism and Head Piece Including Same

(75) Inventor: Frank Albert Bilan, Newman, CA (US)

Correspondence Address: GIRARD & EQUITZ LLP, 1539 Taraval St., SUITE 202, SAN FRANCISCO, CA, 94116, US

(73) Assignee: SILUTIONS TECHNOLOGIES, INC., San Jose, CA (US)

(21) Appl. No.: 12/146,631

(22) Filed: Jun. 26, 2008

(51) Int. Cl. B60T 13/04; F16D 63/00; A61B 7/02

(52) U.S. Cl. 188/166; 188/82.84; 181/131

(57) ABSTRACT

An asymmetric adjustment mechanism, a head piece (e.g., stethoscope head piece or audio headset) including such an adjustment mechanism coupled to an ear tip subassembly, and a stethoscope including such a head piece. In typical embodiments, the adjustment mechanism can be closed (e.g., to reduce separation between ear tips) by exertion of closing force on the ear tip subassembly, or opened by exertion of opening force on the ear tip subassembly. In some embodiments, it automatically locks (e.g., with a desired separation between ear tips) upon cessation of user-exerted adjustment force in a locked state in which it exerts no more than an acceptably small (e.g. zero) bias force on the ear tip subassembly. The bias force has magnitude less than that of the opening force required to separate the ear tips to remove them from the user's ears. Preferably, the adjustment mechanism closes in response to relatively low closing force on the ear tip subassembly but requires that the user exert greater opening force to open it. Some embodiments of the adjustment mechanism are configured for use (e.g., as hinges) in or with apparatus other than head pieces, such as, for example, home appliance or motor vehicle doors, non slamming house doors, springless gates, or laptop screens.

### BACKGROUND

1. Field of the Invention

The present invention relates to head pieces (e.g., audio headsets and stethoscope head pieces) and to adjustment mechanisms useful therein for adjusting the separation between ear tips of the head pieces. In a class of embodiments, the invention pertains to an asymmetric adjustment mechanism for holding a pair of ear tips of a head piece (e.g., a stethoscope head piece) against the ears of a user with appropriate pressure to seal against leakage and function adequately in other respects, concomitant with comfort and ease of function.

2. Prior Art

Throughout this disclosure, including in the claims, the expressions “headset” and “head piece” are used as synonyms to denote an apparatus configured to be worn on or positioned against a user's head. Examples of headsets are the head pieces of stethoscopes, audio headphones (of the type that include a small loudspeaker for each ear, to make audible the output of a home or portable audio system), and telephone headsets (of the type including a microphone as well as a small loudspeaker for each ear).

The expression “ear tip” herein denotes each element of a head piece intended and configured to be positioned in, against, or near to an ear of the head piece's user. Examples of ear tips include the small loudspeakers of a pair of audio headphones, and the soft ear tips (which do not contain transducers) of the head pieces of passive stethoscopes.

Throughout this disclosure including in the claims, the expression “active” stethoscope (or “active” sound detection device) denotes a stethoscope (or sound detection device) that includes an acoustic transducer useful for converting acoustic waves (e.g., body sounds of interest) into another form of energy.

Herein, the expression “electronic” stethoscope (or “electronic” sound detection device) denotes a stethoscope (or sound detection device) that includes an acoustic transducer useful for converting acoustic waves of interest (e.g., body sounds) into at least one electric signal. Also herein, the expression “passive” stethoscope (or “passive” sound detection device) denotes a stethoscope (or sound detection device) that does not include an acoustic transducer.

Throughout this disclosure, including in the claims, each of the expressions “acoustic transducer” and “sound transducer” denotes a device for converting acoustic waves into another form of energy. For example, one type of acoustic transducer is a typical microphone configured to convert acoustic waves into an electrical signal. Another example of an acoustic transducer is a device configured to convert acoustic waves into electromagnetic waves (e.g., visible radiation or electromagnetic radiation whose wavelength or wavelengths is or are outside the visible range), and optionally also to convert the electromagnetic waves into an electrical signal. Acoustic transducers are sometimes referred to herein as sound pick-ups, and are sometime referred to herein simply as transducers.

Throughout this disclosure including in the claims, the expression that a first element is “mounted to” a second element denotes that the first element is attached or coupled in any manner to the second element at least one point or region of the second element (each such point or region is denoted herein as a “coupling point”), such that when the second element moves (e.g., vibrates), the first element moves in phase with and in sympathy with the second element at each coupling point. A first element can be “mounted to” a second element if the two elements are directly attached to each other or if they are otherwise coupled to each other (e.g., coupled by any rigid coupling means) without freedom to move relative to each other at each coupling point. A first element can be “mounted to” a diaphragm (a flexible element) at least one coupling point even if portions of the diaphragm other than each coupling point have freedom to move out of phase with the first element.

The expressions “mounted on” and “referenced to” are used herein as synonyms to the expression “mounted to,” with reference to a floating mass that is mounted to a diaphragm.

Throughout this disclosure including in the claims, the expression that a first element is in a “locked” state with respect to a second element does not necessarily mean that the elements are locked with equal force symmetrically, and can denote cases in which they are locked from free movement in one direction more so than another. For example, a hinge can be “locked” from further opening a door, but can easily be moved in the opposite direction to close the door.

Active and passive stethoscopes are used by health care givers (to be referred to herein as physicians since they are typically physicians) to aid in the detection of body sounds for the purpose of diagnosing various symptoms, for example, heart beat anomalies or lung infections. This procedure is commonly called auscultation. Stethoscope design is a specialty art, difficult to learn due to the low sound levels emitted by the body.

A stethoscope includes a head piece which is or includes a pair of ear tips. In use, the ear tips should be positioned in, against, or near to the user's ears with no more than acceptable pressure for user comfort and ease of use. Often, the ear tips need to be positioned in, against, or near to the user's ears with not less than sufficient pressure to seal against sound leakage and/or otherwise enable the stethoscope to function adequately. Other head pieces include ear tips that need to be positioned in, against, or near to the user's ears with no more than acceptable pressure for user comfort and ease of use, and sometimes also with not less than sufficient pressure to seal against sound leakage and/or otherwise function adequately.

Many attempts have been made to improve the comfort and seal efficiency of ear tips of stethoscope headsets and other headsets. However, conventional stethoscope headsets apply spring bias to force the tips into the user's ears, in an effort to create a soundproof seal to maximize the user's ability to hear only the desired sound while excluding outside ambient noise. This can be done quite easily if one disregards comfort, simply by using more pressure against the ears. However, it is unacceptable to disregard user comfort in this context since people's ear canals are very sensitive and also vary in size. On the other hand, conventional implementation of headsets to impose the lightest possible pressure on the ears conflicts directly with ear tip sealing ability.

Herein, the expression “symmetric” head piece denotes a head piece (including ear tips) which, in use with the ear tips in or against the user's ears, exerts inward biasing force against the user's ears, and which requires that force having the same or similar magnitude be exerted but in the opposite directions (i.e., in “outward” directions, away from the user's ears) on the ear tips or ear tip supports to remove the ear tips from the ears. Similarly, the expression “symmetric head piece adjustment mechanism” denotes a mechanism for adjusting at least one of the separation between ear tips of a symmetric head piece and the biasing force exerted by such ear tips on the user's ears during use. Thus, a symmetric head piece having ear tips and a symmetric head piece adjustment mechanism may allow adjustment of the separation between the ear tips, but in use it exerts inward biasing force against the user's ears and requires that force having the same or similar magnitude be exerted (but in opposite, outward, directions) on the ear tips or ear tip supports to remove the ear tips from the ears.

A conventional stethoscope having a symmetric head piece will be described with reference to FIGS. 1 and 2. The stethoscope of FIGS. 1 and 2 is of a type that has been used for decades worldwide, and includes a chest piece 2, a sound carrying tube 3 (made of plastic) including a “Y” section 4, and a head piece coupled to section 4. The head piece consists of ear tip tubes 6, and ear tips 5 at the ends of tubes 6. In FIG. 2 a portion of tube 3 is cut away to show two “U” shaped springs 8 within section 4. Each spring 8 is attached to and coupled between the two ear tip tubes 6. FIG. 2 also shows the open lower ends (7) of tubes 6 inside the open upper ends of tube 3's “Y” section 4. This transition between tube 3 and tubes 6 facilitates the passage of sound picked up by chest piece 2 to ear tips 5.

Pulling outward on ear tip tubes 6 (i.e., pulling the left tube 6 leftward and the right tube 6 rightward in FIG. 2) causes “U” shaped springs 8 to deflect under the strain and exert a biasing force on tubes 6 which in turn causes ear tips 5 to exert inward force on the ears of a physician wearing the stethoscope (i.e., the biasing force causes the tips 5 to press inward against the physicians ears). Springs 8 always exert this biasing force while the physician is wearing the stethoscope. Typically, the force exerted by tips 5 on the physician's ears is in the range from about four to about nine ounces but it can be as high as fourteen ounces in maladjusted stethoscopes. This force against the ears during use of the stethoscope can become very annoying indeed.

Chest piece 2 is generally shaped like a round pill box (as is typical for a chest piece of a conventional stethoscope), with a semi-flexible diaphragm on one side and flexible tube 3 attached to the other side. In use, the diaphragm side of chest piece 2 is placed against the body surface so that sounds from the body cause the diaphragm to move in sympathy. The air space in the interior of chest piece 2 experiences minute pressure waves from the moving diaphragm. These pressure waves travel up flexible tube 3 and tubes 6 and are perceived as sound by the physician's ears as they propagate out from ear tips 5.

The present invention addresses a particularly difficult problem with stethoscope head pieces (and other head pieces): the problem of spring pressure against the user's ears. This irritating pressure can be quite serious, especially when a user is compelled to wear a head piece over a prolonged period (e.g., a pediatrician when examining children all day, or an army doctor when examining hundreds of enlistees every day).

A variety of symmetric head piece adjustment mechanisms have been proposed for use in stethoscopes having symmetric headpieces. For example, U.S. Pat. No. 3,746,124, granted to Wilson, deceased et al., discloses a stethoscope using two leaf springs adjustable up or down the two linear earpiece tubes. This varies the fulcrum point thus achieving variable spring tension.

U.S. Pat. No. 5,561,275, granted to Savage, et al., discloses a stethoscope having a screw adjustable stem piece that exposes or occludes equal portions of a pair of spring-beam head piece members. This provides some tension adjustability.

U.S. Pat. No. 5,920,038, granted to Foster, discloses a stethoscope including a superelastic memory metal arm that is user-adjustable due to the superelastic memory material's characteristics. Adjustment is made by forcing the arm to a desired deflection. When the deflecting force is discontinued, the superelastic memory material “remembers” and returns to its original configuration.

U.S. Pat. No. 6,595,316, granted to Cybulski, et al., describes a very complex mechanism for adjusting the spacing of stethoscope earpieces. The Cybulski stethoscope includes spring blades 30a and 30b, each coupled to a different earpiece (11a or 11b). During use of the stethoscope, spring blades 30 and 30b exert biasing force on the earpieces to spring-bias the earpiece free ends against the user's ears. Each of blades 30a and 30b has a variable seating point around a central fulcrum hub. The seating points are adjustable by changing the orientation of cam 47. Adjusting the seating points changes the spacing between the earpieces' free ends at the user's ears. When cam 47 positions earpieces 11a and 11b in a first configuration with narrow spacing between the earpieces' free ends, and a user displaces the free ends further apart (so that they have a “first” separation) to place them over his ears, spring blades 30a and 30b exert a relatively small biasing force on the earpieces (spring-biasing the earpiece free ends against the user's ears). When cam 47 holds earpieces 11a and 11b in a second configuration with narrower spacing between the earpiece free ends, the user must exert greater bending force on earpieces 11a and 11b to separate their free ends by said first separation and place the free ends over his ears, and when this has been done, spring blades 30a and 30b exert greater bias force on the earpieces, causing them to press inward against the users ears with greater spring force.

Regardless of whether the separation between spring blades 30a and 30b is narrowed or widened, it is necessary to perform a sequence of adjustment steps which include exertion of similar forces on cam 47. Specifically, cam 47 must be unlocked by pushing it perpendicular to the plane of Cybulski's FIG. 4 and then rotated into an orientation that separates blades 30a and 30b as desired. Then cam 47 is released, and returns to a locked state in its new orientation.

It would be desirable to implement a head piece (e.g., a stethoscope head piece) to include a non-symmetric head piece adjustment mechanism capable of adjusting the separation between its ear tips in such a manner that, in use with the ear tips in or against the user's ears, the head piece does not exert inward biasing force against the user's ears (or exerts inward biasing force against the user's ears having magnitude less than the magnitude of the outward force required to separate the ear tips to remove them from the user's ears). Such a head piece, unlike the symmetric head pieces of the Cybulski apparatus and other conventional stethoscopes, would preferably be conveniently adjustable merely by directly pulling apart or pushing together its ear tips or ear tip supports, and would automatically lock the ear tips (upon cessation of the adjusting force) in a configuration that maintains desired inter-tip separation without exerting spring bias force on the ear tips (or by exerting inward biasing force against the user's ears having magnitude less than the magnitude of the outward force required to separate the ear tips to remove them from the user's ears).

### SUMMARY OF THE INVENTION

In a class of embodiments, the invention is a head piece including an ear tip subassembly (including ear tips and typically also ear tip supports that support the ear tips) and a head piece adjustment mechanism coupled to the ear tip subassembly. The head piece adjustment mechanism is typically configured to allow a user to adjust the separation between the ear tips to position the ear tips in or against the user's ears and to hold the ear tips in this position with appropriate pressure to seal against leakage and otherwise function adequately, concomitant with comfort and ease of function. The head piece adjustment mechanism can be “closed” (adjusted so as to reduce the separation between the ear tips, e.g., as when the user places them in or against the user's ears) or “opened” (adjusted so as to increase the separation between the ear tips) by exerting force on the ear tip subassembly. In some embodiments in this class the head piece is a stethoscope head piece. In other embodiments, the head piece is an audio headset. In typical embodiments in which the head piece is an audio headset, each ear tip includes a small loudspeaker configured to convert an electrical signal into sound audible to a physician or other user when the ear tip is positioned in or adjacent to an ear of the user.

The head piece adjustment mechanism is “asymmetric” in the following sense. It is configured to allow the user to close it (reduce the separation between the ear tips) by exerting closing force (in first directions) on the ear tip subassembly, or open it (increase the separation between the ear tips) by exerting opening force (in second directions opposite to the first directions) on the ear tip subassembly, and in response to cessation of the opening or closing force (adjustment force) it automatically enters a locked state (e.g., with a desired separation between ear tips) in which it exerts no more than an acceptably small bias force (e.g. zero or substantially zero spring bias force) on the ear tip subassembly, said bias force having magnitude less than the minimum opening force magnitude required to open the adjustment assembly from its locked state (e.g., to separate the ear tips to remove them from the user's ears). In this context, the expression “closing force” is used in a broad sense to denote either force or torque applied in the “first directions” (which may be directions toward the user's ears), and the expression “opening force” is used in a broad sense to denote either force or torque applied in the “second directions” (which may be directions away from the user's ears). The head piece adjustment mechanism is thus sometimes referred to herein as an “asymmetric” adjustment mechanism or an “asymmetric tension” adjustment mechanism. Preferably, the head piece adjustment mechanism of the inventive head piece is configured to be closed, to reduce separation between the ear tips (e.g., to introduce the ear tips into the ear canals) in response to relatively low closing force (force having less than a threshold magnitude) on the adjustment mechanism in its locked state (e.g., on ear tips or ear tip supports coupled to the adjustment mechanism) but requires the user to exert force having magnitude equal to or greater than the threshold magnitude on the adjustment mechanism in the locked state to open it, to increase separation between the ear tips (e.g., to move ear tips in the ear canals out from the ear canals and away from the user's ears). In such preferred embodiments, the ear tips remain locked with a desired separation between them (e.g., in or adjacent to the user's ears) during use as long as no opening force having magnitude in excess of the threshold magnitude is exerted on the adjustment mechanism, but the ear tips can be further closed (i.e., the separation between them reduced) by exerting closing force having magnitude less than the threshold magnitude on the adjustment mechanism.

In typical use of embodiments of the inventive head piece which include ear tip supports between the ear tips and the asymmetric adjustment mechanism, the user separates the ear tip supports (which are stethoscope ear tip tubes in embodiments in which the head piece is a stethoscope head piece included in a stethoscope) by exerting outward force on them. The user then exerts a smaller inward force (typically no more than gentle pressure) on the supports to move the ear tips toward each other and guide the ear tips into his or her ears so as to achieve a comfortable fit and seal, and then releases hold on the ear tip supports. Upon cessation of inward adjusting force on the supports, the ear tips remain locked in place without the head piece exerting inward biasing force against the user's ears. Specific embodiments configured for use in this manner are described herein.

When in a locked state (e.g., to maintain fixed separation between the free ends of ear tip supports), preferred embodiments of the asymmetric head piece adjustment mechanism remain locked during use unless and until opening force having magnitude in excess of a threshold magnitude is exerted on the adjustment mechanism (or until sufficient closing force is exerted thereon). When so locked, the adjustment mechanism can easily be further closed (e.g., temporarily unlocked and moved into a configuration with reduced separation between the free ends of ear tip supports attached to the adjustment mechanism) by exerting relatively small closing force thereon (e.g., closing force having magnitude less than the threshold magnitude) and will automatically re-lock upon cessation of the closing force.

During use of a typical embodiment of the inventive head piece (with ear tips of the head piece in or against the user's ears), the head piece does not exert spring biasing force that biases the ear tips against the user's ears (or it exerts substantially no spring biasing force). Rather, the adjustment mechanism locks the ear tips in the desired position without biasing them against the user's ears and without exerting constant spring biasing force on the ear tips or ear tip supports. Alternative embodiments of the head piece include an adjustment mechanism that can lock the ear tips in a desired position in or against a user's ears while the device also biases (e.g., while spring arms of the device bias) the ear tips against the ears. Preferably, the ear tips exert relatively low (e.g., zero) force on the user's ears when the ear tips have been placed in a desired position in or against a user's ears (e.g., when the ear tips have been introduced into the ear canals).

In another class of embodiments, the invention is an asymmetric adjustment mechanism (e.g., an asymmetric hinge mechanism) for adjusting relative orientation of first and second members. In typical embodiments in this class, the mechanism is configured for use as a head piece adjustment mechanism in a stethoscope head piece or other head piece, and the first and second members are ear tip supports or ear tips. Alternatively, the adjustment mechanism is configured for use as an asymmetric adjustment mechanism (e.g., an asymmetric hinge mechanism) in another system or apparatus. For example, some embodiments are configured for use in or with a home appliance door (e.g., a springless oven door, a springless horizontal refrigerator or freezer door, or another springless refrigerator or freezer door), an automobile (or other motor vehicle) door, a non slamming house door, a springless gate, or a laptop screen. Herein, “laptop” denotes a laptop or notebook computer, tablet PC, PDA (personal digital assistant), or other portable computer or computing system. For example, the door, gate, or screen can be mounted on one or more of the inventive adjustment assemblies, with each adjustment assembly serving as a hinge. For example, a door, gate, or screen mounted on such a hinge (or hinges) can be opened by applying relatively small force thereto or closed by applying relatively large force thereto, and will remain locked in a user-determined orientation upon cessation of opening or closing force thereon. In preferred embodiments in this class, the asymmetric adjustment mechanism is a spring clutch device or includes a roller clutch bearing (or other cylindrical clutch bearing).

In many different embodiments of the invention (including typical stethoscope embodiments), the asymmetric adjustment mechanism is preferably a spring clutch device. In some other embodiments, the asymmetric adjustment mechanism includes a roller clutch bearing.

The inventive spring clutch device includes a shaft, a locking spring around the shaft, and a control assembly movable between positions in engagement with the locking spring. In response to locking torque (exerted by the control assembly on at least one portion of the locking spring), the locking spring tightens against the shaft (and is thus locked, at least temporarily, with respect to the shaft). In typical embodiments, continuous application of locking torque (having magnitude greater than a threshold magnitude) to the locking spring causes the locking spring to sequentially lock and then unlock relative to the shaft. This sequence includes a number of repetitions of the following steps: the locking torque initially locks the locking spring with respect to the shaft, and then it temporarily unlocks the locking spring (to allow the locking spring to rotate temporarily relative to the shaft into another locking state in which it is again locked relative to the shaft). In response to unlocking torque (exerted by the control assembly on at least one portion of the locking spring), the locking spring loosens relative to the shaft so that it is free to rotate around the shaft (e.g., in response to closing force exerted on the spring clutch device).

In other embodiments, the invention is an assembly including a member, and an adjustment mechanism coupled to the member and configured as a hinge that allows clockwise rotation of the member relative to the adjustment mechanism in response to closing force, on the member and the adjustment mechanism, of magnitude in excess of a threshold magnitude but not in response to closing force of magnitude less than the threshold magnitude. The hinge also allows counterclockwise rotation of the member relative to the adjustment mechanism in response to opening force on the member and adjustment mechanism of magnitude in excess of a second threshold magnitude but not in response to opening force of magnitude less than the second threshold magnitude, where the second threshold magnitude is different than the threshold magnitude. In some embodiments, the hinge assembly also includes a second member coupled to the adjustment mechanism such that the second member has freedom to rotate clockwise relative to the member in response to closing force (on the member and second member) of magnitude in excess of the threshold magnitude but not in response to closing force of magnitude less than the threshold magnitude, and to rotate counterclockwise relative to the member in response to opening force (on the member and second member) of magnitude in excess of the second threshold magnitude but not in response to opening force of magnitude less than the second threshold magnitude. In some embodiments, at least one of the threshold magnitude and second threshold magnitude is adjustable during at least one of manufacture, configuration, and use of the adjustment mechanism. In some embodiments, the adjustment mechanism is a slip clutch.

In another class of embodiments, the invention is an assembly, including a member and an adjustment mechanism coupled to the member, where the assembly is configured as a hinge such that the member is rotatable clockwise and counterclockwise relative to the adjustment mechanism, at least a minimum force must be exerted on the adjustment mechanism to rotate the member clockwise relative to the adjustment mechanism, and the minimum force is asymmetric with respect to a second minimum force required to be exerted on the adjustment mechanism to rotate the member counterclockwise relative thereto. In some such embodiments, at least one of the minimum force and the second minimum force is adjustable during at least one of manufacture, configuration, and use of the assembly.

In another class of embodiments, the invention is an adjustment mechanism including a first assembly and a second assembly coupled to the first assembly such that the first assembly is rotatable clockwise and counterclockwise relative to the second assembly. The adjustment mechanism is configured such that at least a minimum force must be exerted on the adjustment mechanism to rotate the first assembly clockwise relative to the second assembly, and the minimum force is asymmetric with respect to a second minimum force required to be exerted on the adjustment mechanism to rotate the first assembly counterclockwise relative to the second assembly. In some such embodiments, the first assembly and the second assembly together implement a spring clutch device, or a roller clutch bearing or cylindrical clutch bearing is included in one of both of the first assembly and the second assembly.

In another class of embodiments, the invention is a head piece, including an ear tip subassembly including ear tips, and an adjustment mechanism coupled to the ear tip subassembly and configured to close to reduce separation between the ear tips in response to closing force on the ear tip subassembly having magnitude greater than a threshold magnitude but not in response to closing force having magnitude less than the threshold magnitude, and to open to increase separation between the ear tips in response to opening force on the ear tip subassembly having magnitude greater than a second threshold magnitude but not in response to opening force having magnitude less than the second threshold magnitude, where the second threshold magnitude is different than the threshold magnitude. In preferred embodiments in this class, the adjustment mechanism's dominant characteristic of opening is non energy storing (as defined hereinbelow) and/or adjustment mechanism's dominant characteristic of closing (as defined hereinbelow) is non energy storing.

In a class of embodiments, the invention is a stethoscope including a chest piece (including a diaphragm and optionally also an acoustic transducer mounted to sense movement of the diaphragm), and a head piece coupled (e.g., by a tube) to the chest piece. The head piece includes ear tips, an adjustment mechanism, and ear tip tubes coupled between the ear tips and the adjustment mechanism. The adjustment mechanism can be of any of the types described herein. Typically, the adjustment mechanism is configured to be “closed” in response to user movement of the ear tip tubes which reduces separation between the ear tips (e.g., as when the user places them in or against the user's ears) and to be “opened” in response to user movement of the ear tip tubes which increases separation between the ear tips. Typically, the adjustment mechanism is an asymmetric mechanism in the sense that it is configured to allow the user either to close it by exerting closing force (in first directions) on the ear tips or ear tip tubes, or open it by exerting opening force (in second directions opposite to the first directions) on the ear tips or ear tip tubes, but it automatically locks the ear tips (upon cessation of the user-exerted adjustment force) with a desired separation between them and without exerting spring bias force on the ear tips (or with exertion of no more than an acceptably small inward bias force on the ear tips, having magnitude less than the magnitude of the outward force required to separate the ear tips to remove them from the user's ears). Preferably, the adjustment mechanism is configured so that the user can close it by moving the ear tip tubes together (to reduce separation between the ear tips) by exerting relatively low force (i.e., force having less than a threshold magnitude) on the ear tip tubes, but so that it cannot be opened in response to user-exerted opening force on the ear tip tubes (which moves the ear tip tubes away from each other to increase the separation between the ear tips) unless the opening force has a magnitude not less than the threshold magnitude.

In some embodiments of the inventive stethoscope, the stethoscope is an active stethoscope including an acoustic transducer in the chest piece, and at least one output transducer (e.g., an output transducer at each ear tip). Typically, the acoustic transducer generates at least one electrical signal in response to body sounds that cause the diaphragm to move, and each output transducer is coupled and configured to convert at least one said electrical signal into sound that is audible to a physician or other user. In other embodiments in this class, the acoustic transducer produces a transducer output in response to body sounds that cause movement of the diaphragm, and the stethoscope includes headset driving circuitry coupled to the acoustic transducer and each output transducer. The headset driving circuitry is configured to generate at least one electrical signal in response to the transducer output (which may, for example, be an optical signal), and each output transducer is coupled and configured to convert at least one said electrical signal into sound that is audible to a user.

### BRIEF DESCRIPTION OF THE DRAWINGS

FIG. 1 is an elevational view of a conventional stethoscope.

FIG. 2 is an elevational view, partially cut away, of a portion of the stethoscope of FIG. 1.

FIG. 3 is an elevational view, partially cut away, of a portion of a first embodiment of the inventive stethoscope.

FIG. 3B is an elevational view, partially cut away, of an implementation of an ear tip 10 and a portion of a head piece tube 16 of the FIG. 3 stethoscope.

FIG. 4a is a perspective view of roller clutch bearing 20 and shaft 21 of the embodiment of the invention shown in FIGS. 3 and 5, with shaft 21 coaxial with but separated from bearing 20.

FIG. 4b is a cross-sectional view (along line A-A of FIG. 4a) of roller clutch bearing 20 and shaft 21 of FIG. 4a, with shaft 21 positioned within (surrounded by) bearing 20.

FIG. 5 is an elevational view of a portion of the FIG. 3 embodiment, including pivot mechanism 18.

FIG. 6 is an exploded perspective view of an implementation of the FIG. 5 assembly (without tubes 16).

FIG. 7 is an elevational view, partially cut away, of a portion of a second embodiment of the inventive stethoscope.

FIG. 8 is a perspective view of a first embodiment of the inventive spring clutch mechanism.

FIG. 8a is a perspective view of a second embodiment of the inventive spring clutch mechanism.

FIG. 9 is a perspective view of a third embodiment of the inventive spring clutch mechanism.

FIG. 9a is a perspective view of a portion of a variation on the FIG. 9 embodiment.

FIG. 10 is a perspective view of spring clutch mechanism 48 of FIG. 7.

FIG. 11 is an exploded perspective view of a portion of the FIG. 10 assembly.

FIG. 12 is an enlarged perspective view of a portion of spring clutch mechanism 48 of FIG. 10.

FIG. 13 is a perspective view (partially in phantom view) of another embodiment of the inventive asymmetric adjustment mechanism.

FIG. 14 is an elevational view, partially cut away, of a portion of an embodiment of the inventive stethoscope which includes a light closing bias spring.

FIG. 15 is an elevational view, of an embodiment of the inventive adjustment mechanism in a headset application.

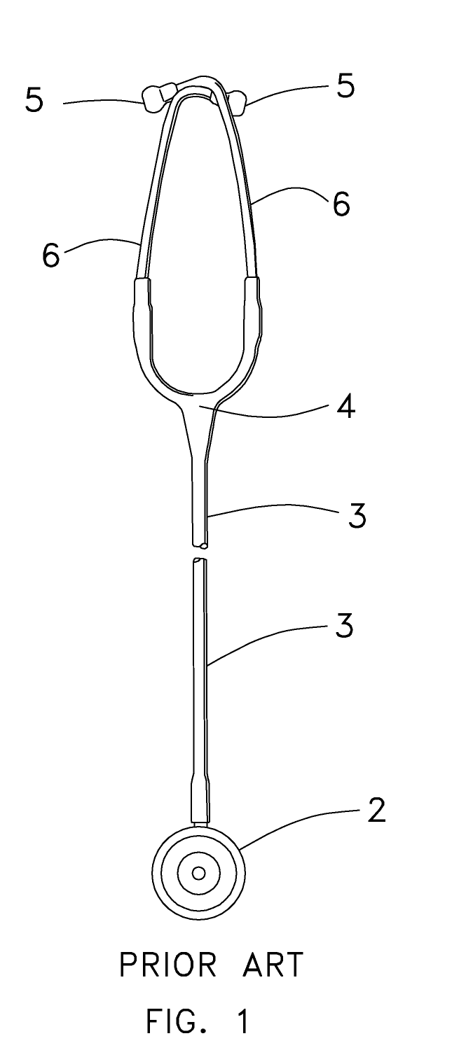

### DETAILED DESCRIPTION OF THE PREFERRED EMBODIMENTS

Many embodiments of the present invention are technologically possible. It will be apparent to those of ordinary skill in the art from the present disclosure how to implement them.

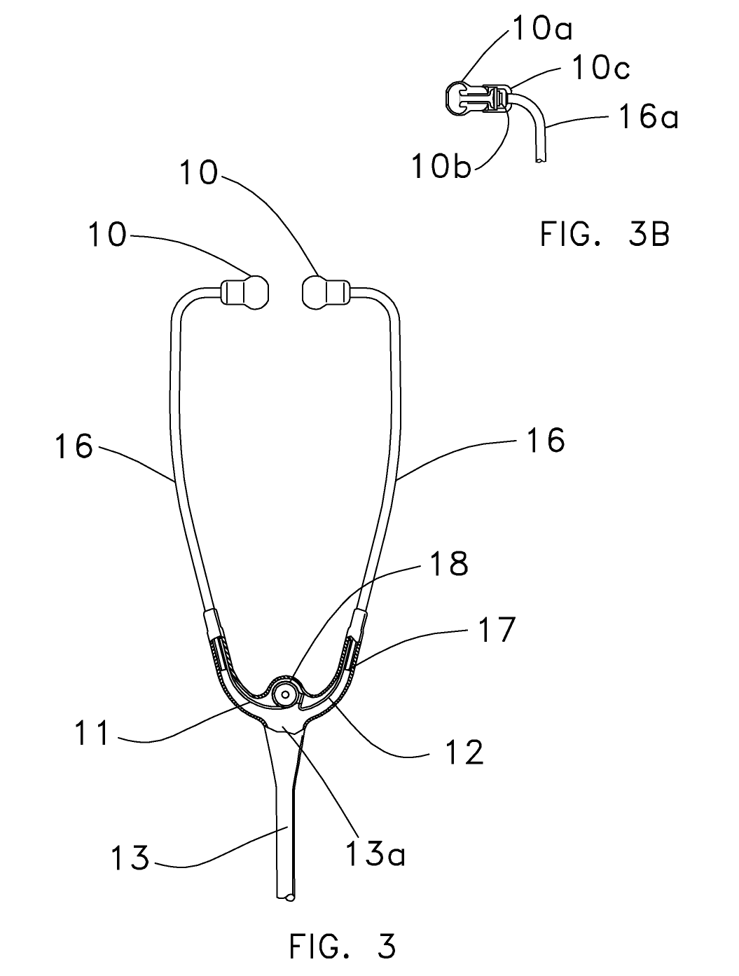

A first embodiment of the inventive stethoscope is shown in FIG. 3. The stethoscope of FIG. 3 includes a sound carrying tube 13, which has a chest piece (not shown) attached to its lower end and a “Y” section 13a at its upper end. The stethoscope of FIG. 3 also includes a head piece coupled to section 13a. The head piece includes head piece tubes 16 (sometimes referred to herein as ear tip tubes 16), and ear tips 10 at the ends of tubes 16.

FIG. 3B is a partially cut away view of an implementation of an ear tip 10 and a portion of a head piece tube 16 of the FIG. 3 stethoscope. To implement the FIG. 3 stethoscope as an electronic stethoscope, each ear tip 10 can be implemented to include an end portion 10a (for placement in the meatus of a user's ear), a sound transducer 10b, and a housing 10c coupled to an end portion 16a of earpiece tube 16. In a typical electronic stethoscope implementation, tube 16 would include necessary signal wires (not shown) extending between the stethoscope's electronics (which generate an electrical signal indicative of detected acoustic waves) and sound transducer 10b (which generates output sound audible to the user in response to the electrical signal).

FIG. 3 shows a portion of sound carrying tube 13 cut away to show an embodiment of the inventive coupling mechanism (11, 12, and 18) coupled between the lower ends of tubes 16, and the open lower ends (17) of tubes 16 inside the open upper ends of “Y” section 13a of tube 13. This transition between tube 13 and tubes 16 facilitates the passage of sound picked up by the chest piece (not shown) to ear tips 10.

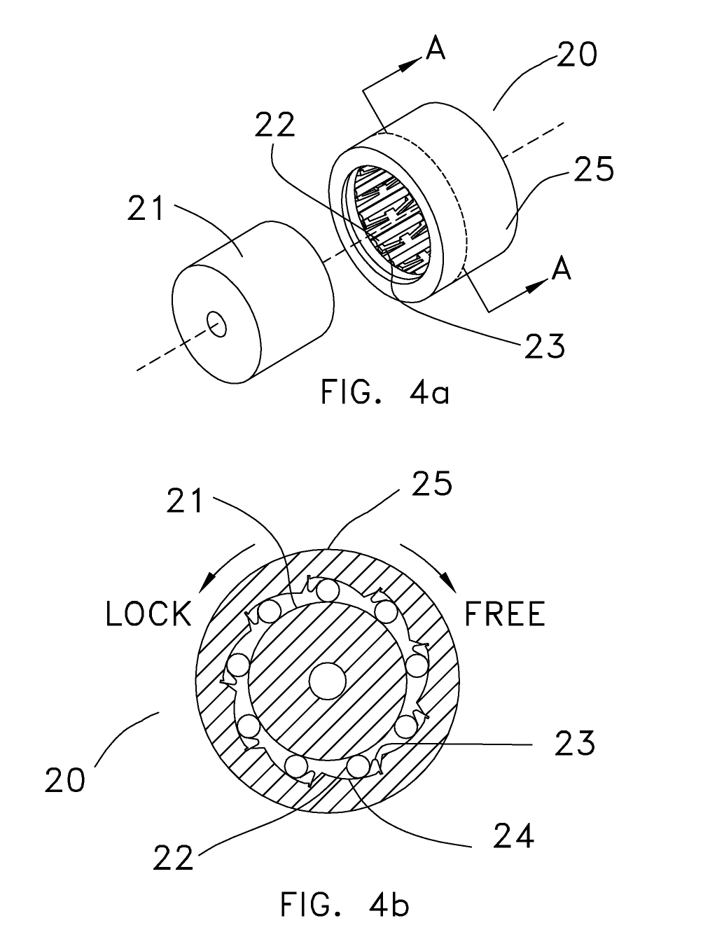

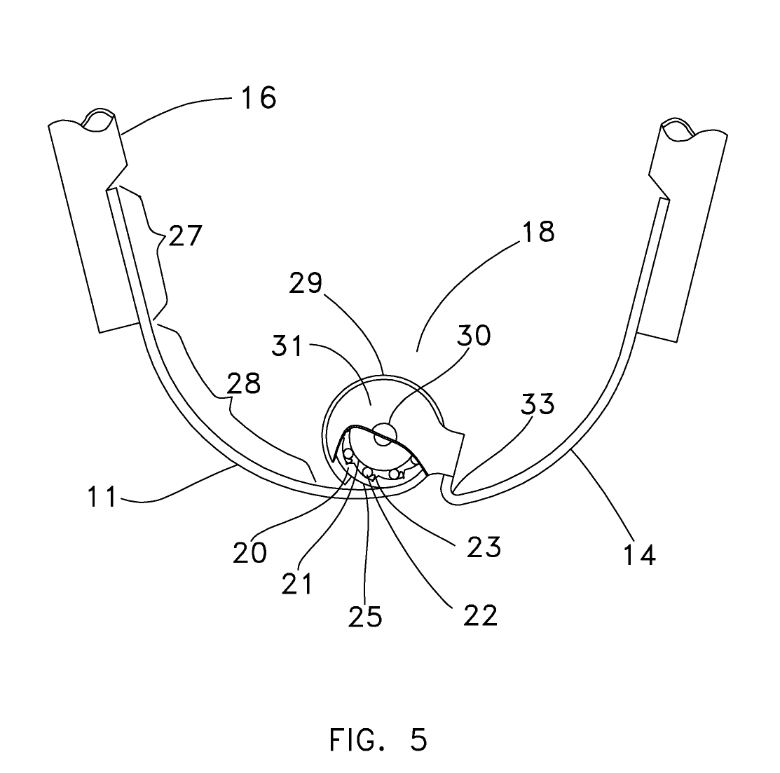

Arms 11 and 12 extend out from pivot mechanism 18 and are attached to the ear tip tubes 16. Pulling outward on the two head piece tubes 16 (i.e., pulling the left tube 16 leftward and the right tube 16 rightward in FIG. 3) causes pivot mechanism 18 (sometimes referred to herein as “pivot” 18) to allow counter-clockwise rotational movement of arm 11 and clockwise rotational movement of arm 12 with a predetermined first friction. Since arms 11 and 12 are attached to ear tip tubes 16, such pulling of tubes 16 allows the ear tips 10 to open outward from each other. When ear tips 10 are sufficiently separated from each other, the physician can then place the ear tips 10 into his ears. Movement of ear tips 10 toward each other (into the physician's ear canals) causes tubes 16 to reverse the rotation of pivot 18. The pivot 18 allows the latter movement with no friction (or no more than an insignificant amount of friction relative to the first predetermined friction) because it contains a one way roller clutch bearing 20 as shown in FIGS. 4a, 4b, and 5.

Standard commercial roller clutch bearing 20 of FIG. 4a is suitable for use in some embodiments of the invention. Shaft member 21, useful with bearing 20 is also shown in FIG. 4a. In use, shaft 21 would be positioned within roller clutch bearing 20 (as shown in FIG. 4b) but it is separated from bearing 20 in FIG. 4a to expose the inner workings of the bearing 20. FIG. 4b is a cross-sectional view of roller clutch bearing 20 and shaft 21 of FIG. 4b, with shaft 21 positioned within (coaxial with) bearing 20. Each spring member 23 of bearing 20 is shown having a simple “V” shape in FIG. 4b, to simplify the figure.

In its simplest form, roller clutch bearing 20 comprises housing hub 25, an inner race of cylindrical rollers 22, and spring members 23 that bias the rollers 22 in one direction. Referring to FIG. 4b, the inner annular surface of the housing 25 is machined with miniature inclined planes 24. A different one of the rollers 22 rides on each of the planes 24. In use, rollers 22 do not spin as in a conventional roller bearing since there is not any smooth roundness in the inner annular surface.

When round shaft 21 is placed into roller clutch bearing 20 as depicted in FIG. 4b, two modes of operation are possible: (a) clockwise, and (b) anti-clockwise. In the following description of FIG. 4b, we assume that the shaft 21 is fixed to prevent rotation. In this case, when the hub 25 is rotated clockwise, hub 25 is free to move because the rollers 22, which experience some freedom due to the inclined planes 24, allow this motion. The biasing springs 23 give a little, and slippage between the hub 20 and the stationary shaft 21 occurs. The device is typically implemented so as to encourage rollers 22 to rotate somewhat in this situation, but it does not have to be so implemented because even if it is not, slippage occurs nevertheless. In practice, since the roller clutch bearing is lubricated, the slippage is of negligible friction.

If an attempt is made to rotate the hub 25 in an anti-clockwise direction (with shaft 21 fixed to prevent rotation), a different set of circumstances occur. The inclined planes 24 tend to constrict the clearance of the rollers 22 assisted by the spring members 23 so that immediate lockup occurs between the shaft 21 and hub 25. This action is very positive and within the roller clutch's torque rating, very reliable.

Referring to FIG. 5, a portion of a first embodiment of the inventive stethoscope ear piece section is shown, with sound carrying plastic tube 13 (shown in FIG. 3) omitted from FIG. 5 for clarity. Left arm 11 of the FIG. 5 assembly is a relatively strong, spring steel piece (flat-stock) having a straight end section 27, followed by a lazy bend section 28, followed by a tight circular bend section 29 around the roller clutch bearing 20. In alternative embodiments, arm 11 is made of suitable metal (or non-metallic material) other than spring steel (flat-stock). With reference to FIG. 5, central shaft 21 is placed in the center of roller clutch bearing 20, and is affixed to yoke 31 by fastener 30, so shaft 21 does not rotate.

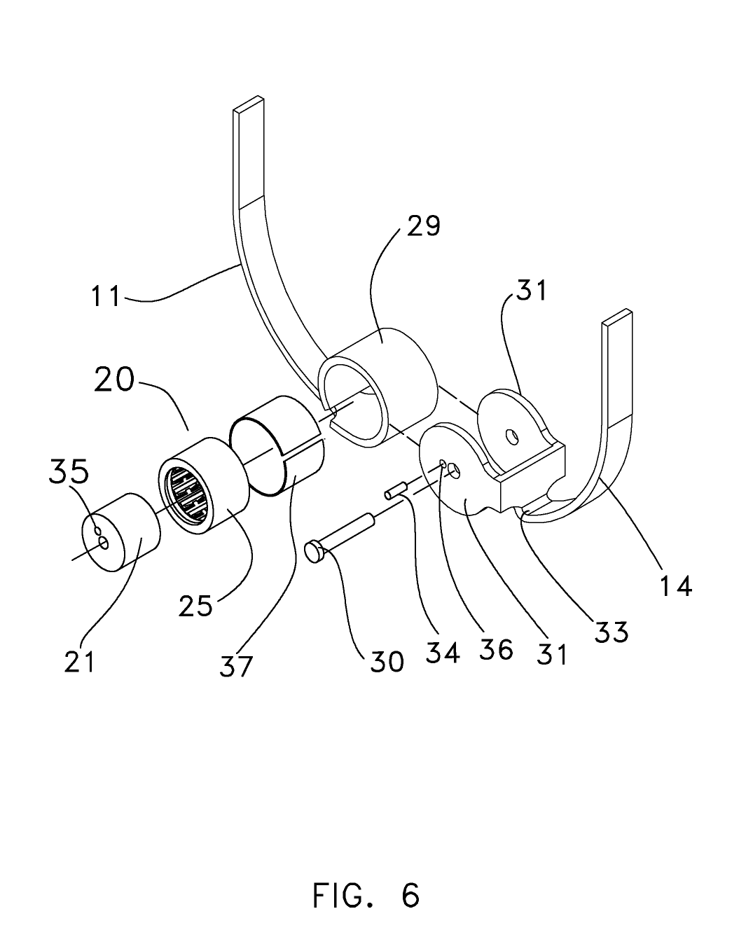

Right arm 14 of the FIG. 5 assembly is made of a conventional steel flat-stock and is similarly shaped to the left arm 11 with the exception that its lower portion is first bent into a sharp angle 33 and terminates at yoke structure 31. Yoke 31 can be integrally formed with the rest of arm 14, or it can be fixedly attached thereto. Arm 11's tight circular bend 29 around the roller clutch bearing 20 is designed to grip hub 25 (which houses roller clutch bearings 22) with considerable force. FIG. 6 shows an implementation of the basic assembly of FIG. 5 in exploded view. In alternative embodiments, arm 14 is made of suitable metal (or non-metallic material) other than spring steel (flat-stock).

Optionally, to achieve smoother clutch action, a friction bearing element (such as, for example, ring 37 shown in FIG. 6) made of plastic or composite material (or other friction bearing material) is included as an interface between circular bend portion 29 and hub 25.

Assembly of the FIG. 5 apparatus is a simple process. Roller clutch bearing 20 is first pressed into the left arm tight circular bend 29 of arm 11 (with or without inclusion of friction bearing material 37 between bend 29 and hub 25) and pivot shaft 21 is then inserted into hub 25 of bearing 20. Yoke portion 31 (at which right arm 14 terminates) is then slipped over circular bend 29 and a fastener 30 is then used to lock the pieces together, forming a pivot mechanism 18 as shown in FIGS. 3 and 5. An exemplary anti-turn lock pin 34 may then be pressed into a drilled hole 36 in yoke 31 and subsequently into punched hole 35 of pivot pin 21, to positively prevent rotation of pivot shaft 21 relative to yoke 31.

In operation, movement of left arm 11 with respect to right arm 14 is now asymmetric in the forces required. When the arms 11 and 14 are moved outward from each other, roller clutch bearing 20 locks but slippage occurs between the tight circular bend 29 and the hub 25 of the roller clutch bearing 20 (because the relatively large amount of force exerted by the user slightly unbends bend 29, thus allowing hub 25 to rotate relative to bend 29 and shaft 21). This is equivalent to the physician opening the stethoscope ear tip arms in preparation for placing then into the meatus of his ears.

When the user moves arms 11 and 14 inward (toward each other), the roller clutch bearing 20 releases and free rotation now occurs between the tight circular bend 29 and hub 25 of roller clutch bearing 20 and stationary shaft 21. This is equivalent to the physician closing the stethoscope ear tip arms as he inserts the ear tips into his ears. The ear tips will now remain where the user has positioned them, because there is a relatively high force necessary to open the ear tips, whereas little or no force is necessary to move the ear tips inwardly into the physician's ear canal. What prevents the ear tips moving further inwardly are the opposing forces of the elasticity of the ear tips and the physician's meatus surrounding the ear canals. Thus, asymmetry of forces is achieved and the physician can adjust a comfortable fit without the nagging spring bias pressure.

The FIG. 5 assembly is a hinge assembly including roller clutch bearing 20 and arm 11 and arm 14 (including yoke portion 31 of arm 14) coupled to bearing 20. Bearing (considered alone) is a conventional roller clutch bearing configured to lock tightly in response to opening force exerted on its components 21 and 25 (i.e., counterclockwise force on component 25 and clockwise force on component 20, viewed as in FIGS. 4b and 5), but to allow rotation of component 21 relative to component 25 in response to closing force (exerted in rotational directions opposite to the directions of the opening force) on components 21 and 25. In contrast, arm 11 (i.e., bent portion 29 of arm 11) of the FIG. 5 assembly is coupled to bearing 20 so as to allow counterclockwise rotation of arm 11 relative to arm 14 in response to opening force on arms 11 and 14 (i.e., counterclockwise force on arm 11 and clockwise force on arm 14, viewed as in FIG. 5), and to allow clockwise rotation of arm 11 (and component 25 of bearing 20) relative to arm 14 (and component 21 of bearing 20) in response to closing force on arms 11 and 14. However, the closing force exerted on arms 11 and 14 need only have magnitude in excess of a first threshold (in order for arm 11 to rotate clockwise to arm 14), whereas the opening force exerted on arms 11 and 14 must have magnitude in excess of a second threshold (in order for arm 11 to rotate counterclockwise to arm 14) greater than the first threshold magnitude. In variations on the FIG. 5 embodiment, arm 14 could be integrally formed with component 21 of bearing 20 (or with at least one component of an adjustment mechanism that replaces bearing 20)

The assembly of FIG. 5 is a one way roller clutch, and is also a slip clutch in the sense that it allows counterclockwise rotation of arm 11 relative to arm 14 by more than 360 degrees in response to opening force on arms 11 and 14 (of magnitude not less than the second threshold magnitude), and allows clockwise rotation of arm 11 relative to arm 14 by more than 360 degrees in response to closing force on arms 11 and 14 (of magnitude not less than the first threshold magnitude). If arm 14 is considered an element of the adjustment mechanism of FIG. 5 (e.g., in variations on the FIG. 5 assembly in which yoke 31 is integrally formed with the rest of arm 14), the adjustment mechanism is also a slip clutch in the sense that it allows counterclockwise rotation of arm 11 relative to the adjustment mechanism by more than 360 degrees in response to opening force on arm 11 and the adjustment mechanism (of magnitude not less than the second threshold magnitude), and allows clockwise rotation of arm 11 relative to the adjustment mechanism by more than 360 degrees in response to closing force on arm 11 and the adjustment mechanism (of magnitude not less than the first threshold magnitude).

It should be appreciated that in the FIG. 6 implementation of the FIG. 5 assembly, 360 degree movement of arms 11 and 14 relative to each other is restricted by both the yoke structure 31 and arms 11 and 14. Nevertheless, the assembly of FIG. 5 is capable of greater than 360 degree movement if the yoke 31 and arms 11 and 14 are shaped and positioned so they do not hit against each other as they rotate relative to each other.

In an experiment, a roller clutch bearing hub (identical to hub 25 of roller clutch bearing 20 of FIGS. 4a and 4b) was cut radially with a small slit. The so-modified roller clutch was determined to have a weaker locking characteristic than that of FIGS. 4a and 4b. A headpiece assembly (sometimes referred to herein as the “modified” assembly) including the modified roller clutch was manufactured. The other elements of the modified assembly were the same as those of FIG. 5, except in that bent arm 29 of FIG. 5 was omitted and replaced in the modified assembly with a rigid attachment to the slit hub (outer shell) of the modified roller clutch bearing. The modified assembly functioned well and as intended, but its locking characteristics were determined to be less predictable than those of the FIG. 5 assembly.

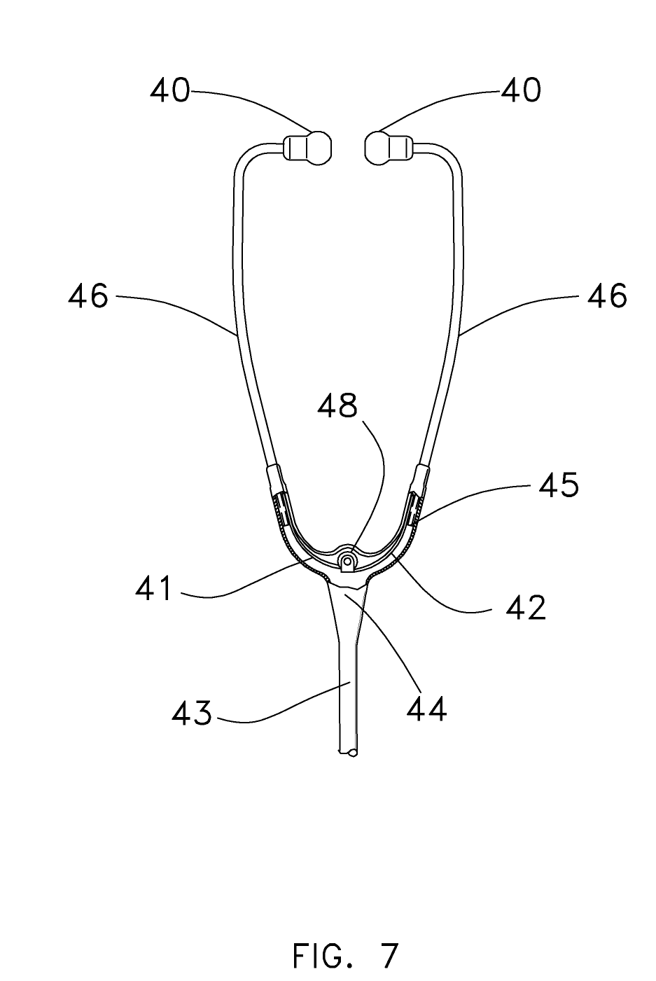

A second embodiment of the inventive stethoscope (and variations thereon) will be described with reference to FIGS. 7-12. As shown in FIG. 7, the stethoscope includes a sound carrying tube 43, which has a chest piece (not shown) attached to its lower end and a “Y” section 44 at its upper end, and a head piece coupled to section 44. The head piece includes earpiece tubes 46 and ear tips 40 at the ends of tubes 46. FIG. 7 shows a portion of sound carrying tube 43 cut away to show an embodiment of the inventive coupling mechanism (41, 42, and 48) coupled between the lower ends of tubes 46, and the open lower ends (45) of tubes 46 inside the open upper ends of “Y” section 44 of tube 43. This transition between tube 43 and tubes 46 facilitates the passage of sound picked up by the chest piece (not shown) to ear tips 40.

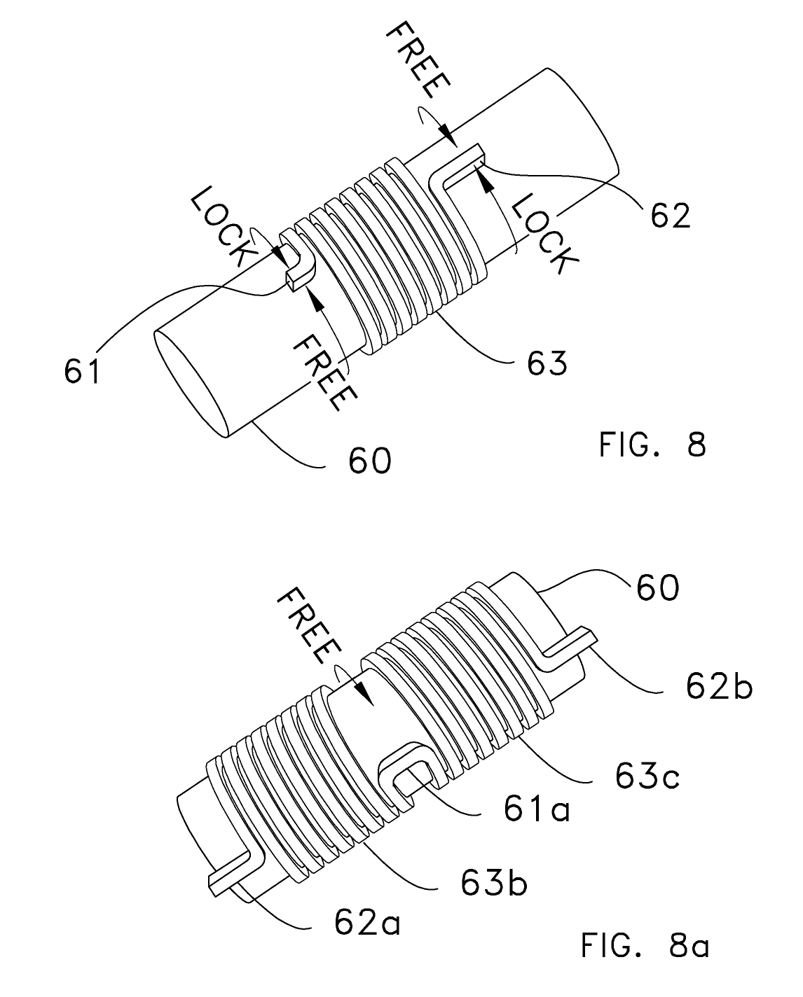

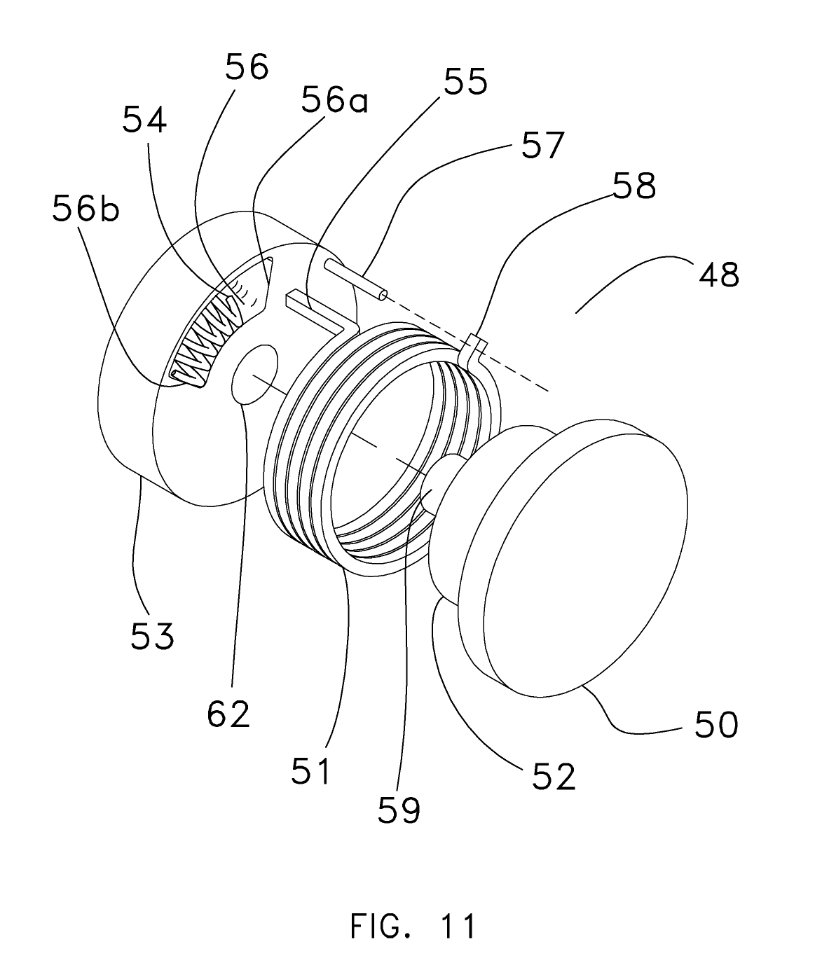

Arms 41 and 42 extend out from pivot mechanism 48 and are attached to the ear tip tubes 46. Pulling outward on the two earpiece tubes 46 (i.e., pulling the left tube 46 leftward and the right tube 46 rightward in FIG. 7) causes pivot mechanism 48 (sometimes referred to herein as “pivot” or “spring clutch mechanism” 48) to allow counter-clockwise rotational movement of arm 41 and clockwise rotational movement of arm 42 with a predetermined first friction. Since arms 41 and 42 are attached to earpiece tubes 46, such pulling of tubes 46 allows the ear tips 40 to open outward from each other. When ear tips 40 are sufficiently separated from each other, the physician can then place the ear tips 40 into his ears. Movement of ear tips 40 toward each other (into the physician's ear canals) causes tubes 46 to reverse the rotation of pivot 48. The pivot 48 allows the latter movement with no friction (or no more than an insignificant amount of friction relative to the first predetermined friction) because it contains an asymmetric spring clutch device 48, shown in exploded view in FIG. 11. Spring clutch mechanism 48 (and variations thereon) and their manner of operation will be described with reference to FIGS. 8-12.

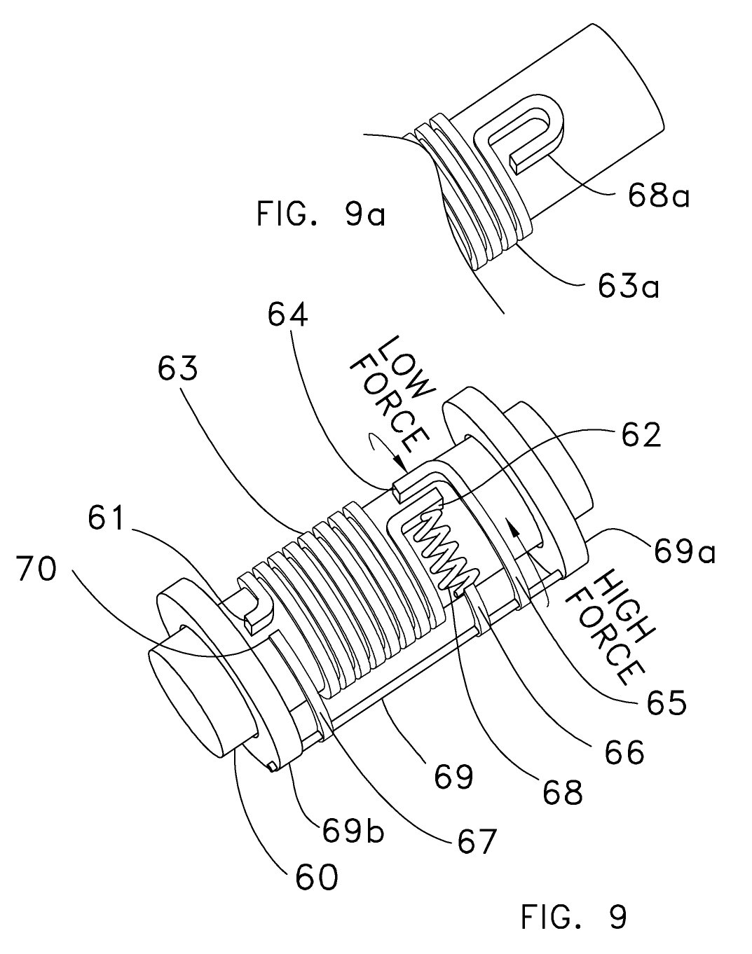

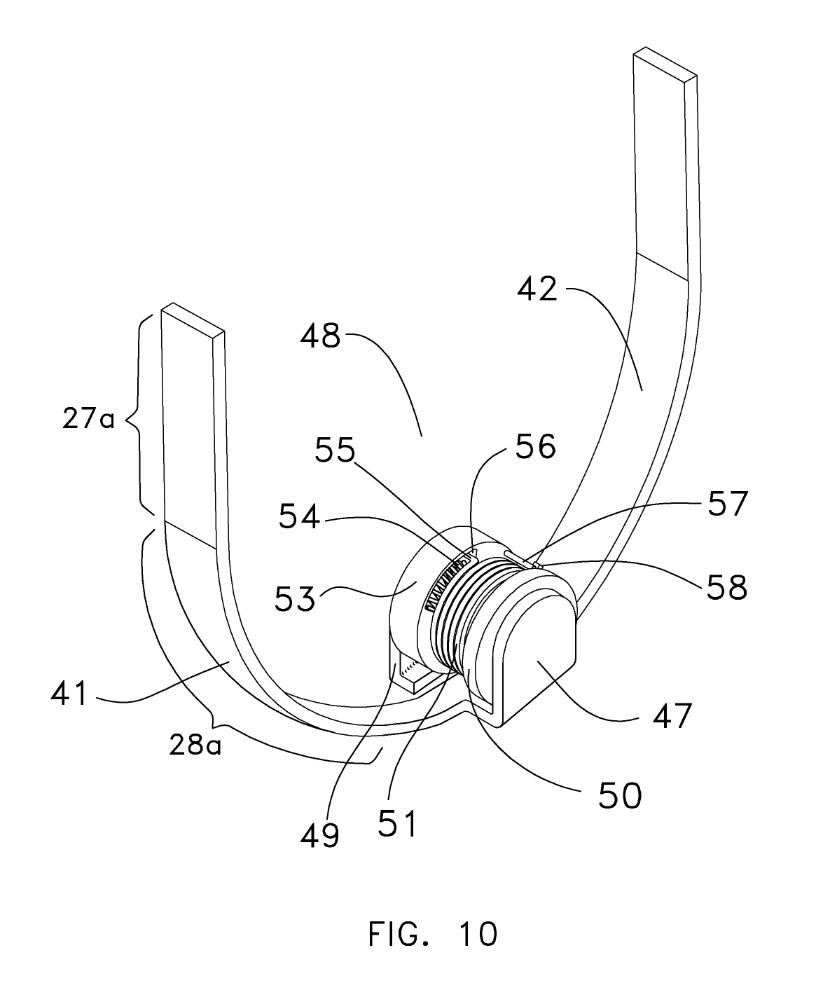

The spring clutch mechanisms of FIGS. 8, 8a, 9, and 9a are exemplary and do not constitute preferred embodiments. A preferred embodiment of spring clutch mechanism 48 will be described with reference to FIGS. 10-12.

The spring clutch mechanism of FIG. 8 (a variation on spring clutch mechanism 48) includes cylindrical shaft 60 and spring 63. Shaft 60 is placed snuggly inside spring 63 as shown, but with spring 63 having very light friction against shaft 60. Typically, spring 63 is made of a square wire stock so that it has maximum contact with shaft 60. To simplify the description, we assume that shaft 60 is held stationary and unable to rotate. Spring 63 has two tangs, 61 and 62, which are bent by 90 degrees relative to the rest of spring 63 so that tangential force (force perpendicular to the longitudinal axis of shaft 60 and tangential to shaft 60's surface) can be applied to them conveniently. Applying a counterclockwise rotational (tangential) force against tang 61 (in the direction of the curved arrow labeled “Free” adjacent to tang 61 in FIG. 8) will cause spring 63 to loosen and hence rotate counterclockwise freely relative to shaft 60. Conversely, applying a force clockwise against tang 61 (in the direction of the curved arrow labeled “Lock” adjacent to tang 61 in FIG. 8) tends to tighten the spring so that it instantly locks against the shaft 60.

Applying a clockwise rotational force against tang 62 at the opposite end of spring 63 (in the direction of the curved arrow labeled “Free” adjacent to tang 62 in FIG. 8) causes spring 63 to loosen and hence rotate clockwise freely. Conversely, applying a force counterclockwise against tang 62 (in the direction of the curved arrow labeled “Lock” adjacent to tang 62 in FIG. 8) tends to tighten the spring so that it instantly locks against the shaft 60.

Note that the two actions described in the two previous paragraphs are opposite in nature, in the sense that on one end of spring 63, free rotation is clockwise while at the other end it is counterclockwise. Similarly, on one end of spring 63, locking rotation is counterclockwise while at the other end it is clockwise.

In the alternative embodiment shown in FIG. 8a, spring 63 of FIG. 8 is replaced by a spring having two portions (63b and 63c) separated by central section 61a. In the balanced spring design of FIG. 8a, spring portions 63b and 63c both are wound clockwise relative to shaft 60 (alternatively, they could both be wound counterclockwise on shaft 60). The spring of FIG. 8a has two tangs, 62a and 62b, at its ends which are bent by 90 degrees relative to the rest of the spring. Freeing rotational force (in the direction of the curved arrow labeled “Free” adjacent to section 61a in FIG. 8a) can be asserted to center section 61a, or locking rotational force (in the same direction) can be asserted equally to tangs 62a and 62b. Those of ordinary skill in the art will recognize that there are many more variations possible on the designs of FIGS. 8 and 8a.

The alternative embodiment shown in FIG. 9 differs from the FIG. 8 embodiment in that it includes two elements in addition to spring 63 and shaft 60: a high compression spring 68; and three arm element 69 including arms 65, 66 and 67. When assembled as shown, the free end of arm 67 is separated by a small distance from the face of spring 63's tang 61, and small, high compression spring 68 has a first end in contact with tang 62 and an opposite end (referred to below as spring 68's “second” end) in contact with the end of arm 66. Element 69 is slidably attached to shaft 60 by rings 69a and 69b (that extend around shaft 60), so that element 69 has freedom to rotate clockwise and counterclockwise around shaft 60 without falling away from shaft 60. Rings 69a and 69b are fixedly attached to element 69's longitudinal rod portion (the elongated portion of element 69 to which arms 65, 66, and 67 are attached). In variations on the FIG. 9 embodiment, element 69 is otherwise mounted to shaft 60 with freedom to rotate clockwise and counterclockwise around shaft 60.

Optionally, arm 66 is fixedly attached to the first end of spring 68. Also optionally, the second end of spring 68 is fixedly attached to tang 62, if spring 68 and element 69 are constructed so that when element 69 is rotated clockwise (in the direction of the curved arrow labeled “Low Force” adjacent to tang 64 in FIG. 9), the force exerted by spring 68 on tang 62 does not prevent tang portion 64 (of arm 65) from impinging on tang 62 and pushing tang 62 clockwise (as described below).

Tang portion (“tang”) 64 at the free end of arm 65 is oriented at an angle of 90 degrees relative to the adjacent portion of arm 65. When the device is assembled as shown, tang 64 is oriented generally parallel to tang 62 of spring 63, and positioned placed very close to and preferably touching the far side of tang 62.

When three arm element 69 is rotated clockwise (in the direction of the curved arrow labeled “Low Force” adjacent to tang 64 in FIG. 9), tang 64 impinges on tang 62 (of spring 63) and pushes tang 62 clockwise, and arm 67 moves away from tang 61. In implementations in which arm 66 is fixedly attached to the first end of spring 68, such clockwise rotation of element 69 also causes arm 66 to pull the first end of spring 68 away from tang 61, without preventing tang 64 from impinging on and pushing tang 62 clockwise. Thus, clockwise rotation of element 69 causes spring 63 to rotate clockwise freely around shaft 60.

When element 69 is rotated counterclockwise (in the direction of the curved arrow labeled “High Force” adjacent to arm 65 in FIG. 9), tang 64 moves away from tang 62 while arm 66 pushes against spring 68 and arm 67 moves toward tang 61. The force exerted by spring 68 on the near face of tang 62 (in response to the force exerted by the advancing arm 66 on spring 68) causes spring 63 to lock against shaft 60. As element 69 then continues to rotate counterclockwise, small spring 68 begins to deflect (its length begins to decrease). As spring 68 compresses between arm 66 and the locked tang 62, the free end 70 of arm 67 begins to approach the near face of tang 61. With even further counterclockwise rotation of element 69 (i.e., in response to locking torque of sufficient magnitude on element 69), a configuration is reached in which end 70 of arm 67 begins to deflect tang 61 counterclockwise, thereby defeating the lock of spring 63 against shaft 60, and causing element 69 and spring 63 to rotate together counterclockwise relative to shaft 60 until compressed spring 68 forces spring 63 to rotate sufficiently to cause the force exerted on tine 61 by arm 67 to decrease sufficiently to relock spring 63 relative to shaft 60. This counterclockwise action is contingent upon the small spring 68 being compressed by a sufficient amount (in response to locking torque of sufficient magnitude on element 69) to allow arm 67 to reach tine 61 to unlock spring 63. Thus, the counterclockwise rotation resistance of the FIG. 9 apparatus (and the threshold magnitude of the locking torque sufficient to trigger its counterclockwise action) is determined by the spring rate of small spring 68. When spring 68 has an appropriate spring rate, the FIG. 9 apparatus functions as an asymmetric rotational clutch device. The described counterclockwise action has mechanical feedback. When tang 61 is pushed by arm 67, the spring 63 begins to move counterclockwise, hence relieving pressure from small spring 68 and re-establishing lock on spring 63. To continue pressure of arm 67 against tang 61, further counter clockwise force is necessary against small spring 68, and so on.

In variations on the FIG. 9 apparatus, spring 68 is not a separate spring. Rather, it is a portion of spring 63 (e.g., a bent portion of tang 62). For example, as shown in FIG. 9a, spring 63 of FIG. 9 can be replaced by spring 63a (of FIG. 9a) which has a U-shaped compression spring portion 68a. Spring portion 68a of FIG. 9a functions as a replacement for tine 62 and separate spring 68 of FIG. 9.

When the spring clutch mechanism of FIG. 9 or 9a is included in a head piece, ear tip supports of the head piece would typically be coupled (indirectly via hub assemblies, or directly) to the spring clutch mechanism so as to exert locking torque on the mechanism's locking spring (spring 63 or spring 63a) in response to user-exerted opening force on the head piece (to separate ear tips of the head piece), and to exert unlocking torque on the mechanism's locking spring in response to user-exerted closing force on the head piece (to move ear tips of the head piece toward each other). Such locking torque would be in the direction labeled “High Force” in FIG. 9, and such unlocking torque would be in the direction labeled “Low Force” in FIG. 9.

A preferred embodiment of spring clutch mechanism 48 will next be described with reference to FIGS. 10-12. FIG. 10 shows this embodiment of mechanism 48, with arms 41 and 42 extending out from mechanism 48's hub assemblies (sometimes referred to herein as hub pieces or hubs) 53 and 50. In typical use, arms 41 and 42 of FIG. 10 would be coupled to ear tip tubes (e.g., ear tip tubes 46 shown in FIG. 7) and the FIG. 10 assembly would be housed within a stethoscope “Y” section (e.g., “Y” section 44 of FIG. 7) at the upper end of a sound carrying tube (e.g., tube 43 of FIG. 7). Left arm 41 of FIG. 10 is preferably a relatively strong steel piece (flat-stock) having a straight end section 27a, followed by a lazy bend 28a, followed by a half yoke piece 47 fastened to one side of hub piece 50 (the near side of piece 50 as viewed in FIG. 10). Right arm 42 of FIG. 10 is preferably a steel piece (flat-stock) similarly shaped to the left arm 41 (with a straight end section, followed by a lazy bend, followed by a half yoke piece 49) with the exception that half yoke piece 49 of arm 42 is a mirrored version of yoke piece 47 of arm 41 and is fastened to hub piece 53 (to the far side of piece 53 as viewed in FIG. 10).

FIG. 11 is an exploded view of the elements of the FIG. 10 assembly other than arms 41 and 42. As shown in FIG. 11, spring clutch assembly 48 operates in the same basic manner as spring clutch assemblies of FIGS. 8, 8a, and 9. Short shaft 52 (protruding from the large diameter end portion of hub 50) of hub 50 of FIG. 11, together with the rest of hub 50, perform the same basic function as does shaft 60 of FIG. 9. Shaft 59 extends out from shaft 52, for coupling the entire hub assembly 50 to hub assembly 53. Spring 51 of FIG. 11 performs the same basic function as does spring 63 of FIG. 9, spring 54 of FIG. 11 performs the same basic function as does spring 68 of FIG. 9, and tangs 55 and 58 of spring 51 of FIG. 11 perform the same basic function as do tangs 61 and 62 of FIG. 9. Round hub assembly 53 has a hole 62 in its inner face for accepting shaft 59 of hub 50.

Preferably, shaft 52 is cylindrical and spring 51 is generally helical, as shown in FIG. 11. In other embodiments of the inventive spring clutch device, the shaft and locking spring have other shapes (e.g., the shaft has a non-circular periphery, and the locking spring wound around the shaft has a cross-sectional shape matching that of the shaft).

Pin 57 (pressed into hub assembly 53) and the rest of hub assembly 53 performs the same basic function as does three arm element 69 of FIG. 9. Hub 53 has a cavity 56 milled therein. Spring 54 is nestled in cavity 56, and tang 55 extends into the space between spring 54 and cavity 56's side wall 56a. Side wall 56a of cavity 56 performs the same basic function as does tang 64 of FIG. 9, and side wall 56b of cavity 56 performs the same basic function as does arm 66 of FIG. 9.

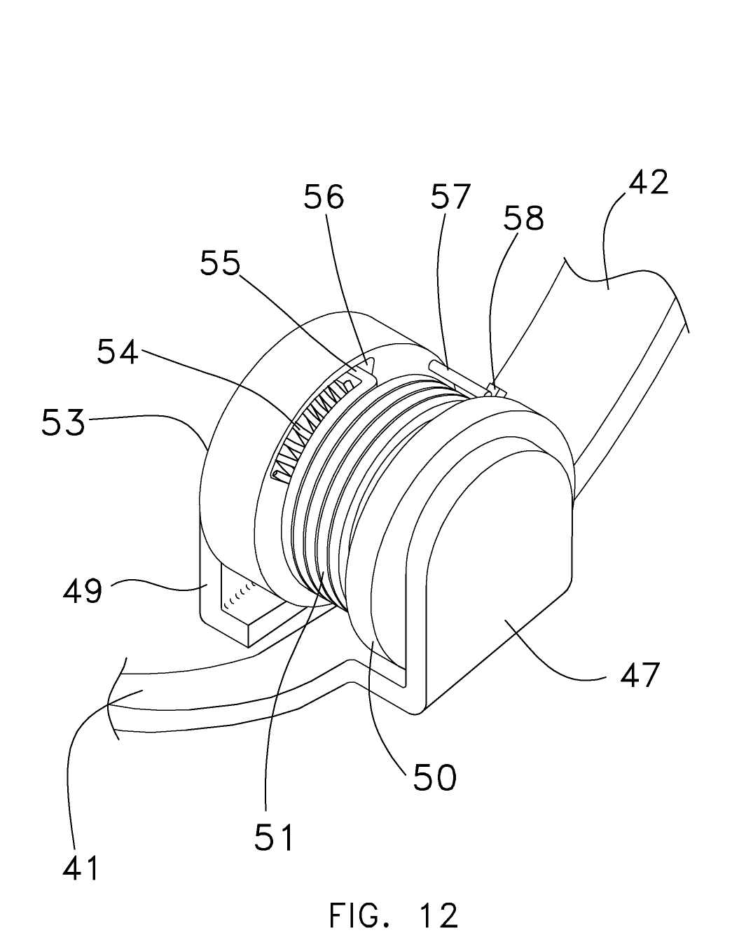

The manner in which spring clutch 48 operates will next be described with reference to FIGS. 10, 11 and 12. Assuming arm 41 (of FIGS. 10 and 12) is held stationary, half yoke 47, hub 50, and shafts 52 and 59 will all be stationary. With arm 41 stationary, counterclockwise rotation of arm 42 applies counterclockwise rotary force (torque) to hub 53. The resulting counterclockwise rotation of hub 53 moves side wall 56a against tang 55. The force exerted by wall 56a on tang 55 tends to open spring 51, causing it to slip freely relatively to shaft 52. This allows arm 42 to continue to rotate toward arm 41, closing the head piece (in which clutch 48 is included) in response to exertion of low closing force by the user. This low, user-exerted, closing force (typically exerted by the user on supports coupled to arm 41 and/or arm 42) causes hub 50 and/or hub 53 to exert unlocking torque on spring 51.

With arm 41 stationary, clockwise rotation of arm 42 applies clockwise rotary force (torque) to hub 53. The resulting clockwise rotation of hub 53 advances one end of spring 54 up against tang 55. The force exerted by the advancing spring 54 on tang 55 tends to close spring 51 causing instant lockup against shaft 52. In this configuration, increasing the clockwise torque on hub 53 compresses spring 54 between wall 56b and tang 55, allowing hub 53 to rotate clockwise a few degrees more. This additional movement of hub 53 causes pin 57 to impinge against the near side of tang 58, thus pushing tang 58 to open spring 51 and defeat its lockup against short shaft 52, in a manner similar to the manner in which arm 67 pushes tang 61 of FIG. 9 to defeat lockup of the FIG. 9 assembly. As clockwise torque continues to be applied to hub 53, spring 54 again compresses against tang 55 which again closes spring 51 (causing instant lockup against shaft 52), and the cycle repeats. This cyclic action (which occurs only in response to locking torque of sufficient magnitude exerted by hub 50 and/or hub 53 on spring 51) allows arm 42 to rotate (stepwise) away from arm 41, opening the head piece (in which clutch 48 is included) in stepwise fashion in response to exertion of relatively high opening force by the user. This stepwise action can be designed to be minute in nature and almost imperceptible, giving the illusion of a smooth action. The relatively high, user-exerted, opening force (typically exerted by the user on supports coupled to arm 41 and/or arm 42) causes hub 50 and/or hub 53 to exert locking torque (of magnitude in excess of a threshold magnitude) on spring 51.

Referring to FIG. 10, movement of the left arm 41 with respect to the right arm 42 is asymmetric in the forces required. When the arms 41 and 42 are moved away from each other (to open a stethoscope in which the FIG. 10 assembly is included), spring clutch 48 cyclically locks and unlocks as described above, allowing relatively high force slippage of clutch 48 (dictated by the relevant parameters of spring 54) which in turn allows arms 41 and 42 to move away from each other in response. This occurs when a physician opens the ear tip arms of a stethoscope in which the FIG. 10 assembly is included, in preparation for placing the ear tips into the meatus of his ears. When the arms 41 and 42 are moved inward toward each other, spring clutch 48 releases and low force rotation now occurs (to close a stethoscope in which the FIG. 10 assembly is included) as described above. This occurs when a physician closes the ear tip arms of a stethoscope in which the FIG. 10 assembly is included, as he inserts the ear tips into his ears. After such insertion of the ear tips into the ears, the ear tips will remain in the ears unless and until a relatively high opening force is exerted to open the arms 41 and 42 (to separate the ear tips). In contrast, relatively little force is necessary to move the ear tips inwardly into the physician's ear canal. What prevents the ear tips moving further inwardly are the opposing forces of the elasticity of the ear tips and the physician's meatus surrounding the ear canals. Thus, asymmetry of opening and closing forces is achieved and the physician can adjust the stethoscope to achieve a comfortable fit without nagging spring bias pressure on his ears.

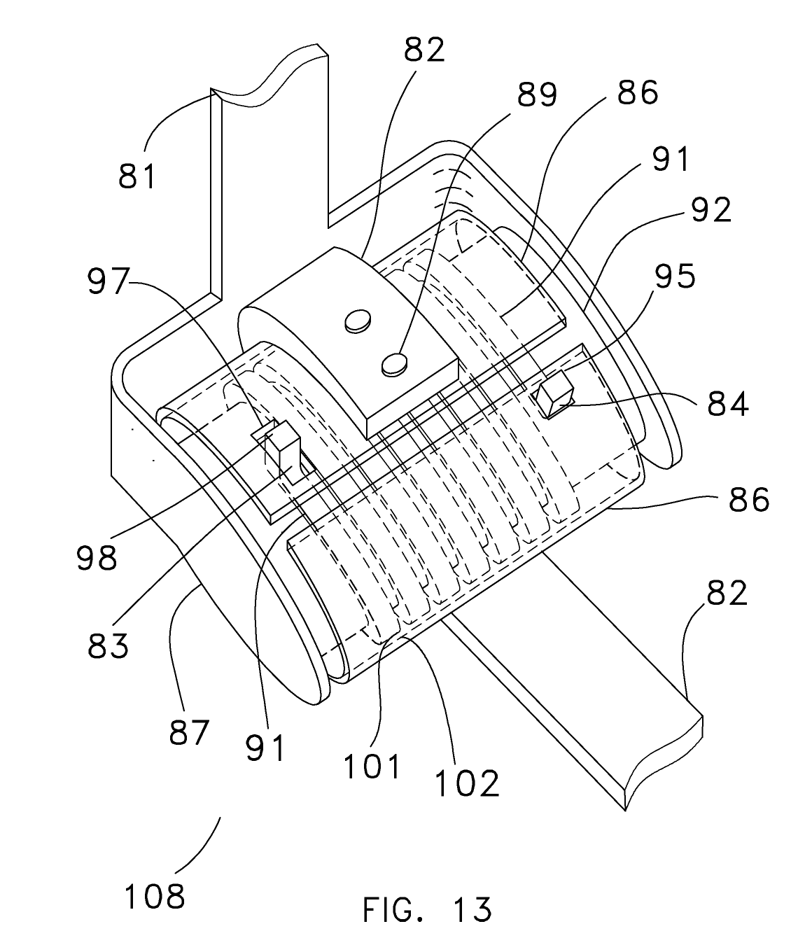

FIG. 13 is a perspective view (partially in phantom view) of another embodiment of the inventive asymmetric adjustment mechanism, which includes a clutch spring 91 wound around shaft 92. This embodiment does not use a small release spring 54 of the type shown in FIGS. 11 and 12. Instead its release spring is a sheet spring 86, made of a sheet of spring material (preferably it is made of spring steel sheet stock). Release spring 86 is shaped to partially surround spring 91 (preferably with a narrow space between springs 86 and 91) with a gap between two opposed edges of spring 86. As shown in FIG. 13, release spring 86 has a generally split cylindrical shape and is thus sometimes referred to herein as “cylinder spring” 86. Cylinder spring 86 has two openings 83 and 84 which extend therethrough, and is positioned so that tangs 95 and 98 of spring 91 protrude radially out through openings 84 and 83 respectively. Deformation of release spring 86 (which causes translation of opening 83 relative to spring 91) in response to force exerted thereon by arm 82 varies the width of the gap between spring 86's two opposed edges. In response to cessation of this deforming force, spring 86 relaxes into a relaxed state in which it locks spring 91 relative to shaft 92.

As shown in FIG. 13, asymmetric adjustment mechanism 108 (sometimes referred to as “spring clutch” 108) comprises arm 82, arm 81 (which terminates in yoke portion 87), and shaft 92 which is fixedly fastened to yoke 87, as well as clutch spring 91 wound around shaft 92. Generally helical spring 91 has two tangs 95 and 98 at its opposite ends. Cylinder spring 86 is held in place around spring 91 by the curved end of arm 82, with tangs 95 and 98 of spring 91 protruding radially out, respectively, a short distance through openings 84 and 83 in spring 86. Opening 84 is small enough that tang 95 fits snuggly therein. Opening 83 is an elongated slot having sufficient length so that tang 98 can translate circumferentially relative to opening 83 and spring 86 while protruding through opening 83.

Cylinder spring 86 is mounted over the clutch spring 91 with a slight gap between them as shown, such that point 101 on spring 91's outer surface is separated slightly from point 102 on spring 86's inner surface. The curved end of arm 82 is wrapped around cylinder spring 86 as shown and fastened to cylinder spring 86 with pins 89.

With reference to FIG. 13, the manner in which spring clutch 108 operates will next be described with the simplifying assumption that arm 81 is held stationary, so that yoke 87 and shaft 92 also remain stationary. In response to application of counterclockwise torque about the axis of shaft 92 to arm 82 (by moving the right end of arm 82 upward as viewed in FIG. 13) the side wall of opening 84 pushes tang 95 (toward the upper left corner of FIG. 13). This tends to release spring 91 under the loosely fitting cylinder spring 86, to allow spring 91, spring 86, and arm 82 to rotate freely around shaft 92. This allows arm 82 to continue to rotate toward arm 81 (e.g., to close a head piece in which mechanism 108 is included) in response to exertion of low opening force by the user.

In response to application of clockwise torque to arm 82, the side wall of opening 84 pushes tang 95 toward the lower right corner of FIG. 13. This tends to close the spring 91 under the loosely fitting cylinder spring 86, causing immediate lockup of spring 91 against shaft 92. Continued application of clockwise torque (about the axis of shaft 92) to arm 82 causes cylinder spring 86 to deform in the sense that it begins to close in around spring 92 (reducing the gap between its opposed edges). This deformation of spring 86 moves wall 97 of opening 83 against tang 98 (i.e., against tang 98's back face, as viewed in FIG. 13), temporarily defeating the lock of spring 91 against shaft 92 and allowing arm 82 to begin to move clockwise away from arm 81. As clockwise torque continues to be applied to arm 82, the side wall of opening 84 again pushes tang 95 toward the lower right corner of FIG. 13 which again closes spring 91 (causing instant lockup against shaft 92), and the cycle (locking of spring 91 followed by temporary unlocking of spring 91) repeats. This cyclic action allows arm 82 to rotate (intermittently) away from arm 81, e.g., to open a head piece (in which mechanism 108 is included) in response to exertion of relatively high opening force by the user.

Arms 81 and 82 are typically attached to stethoscope ear tip tubes (and thus, indirectly to ear tips at the ends of the tubes) in the same manner that arms 11 and 14 of the FIG. 5 assembly are attached to ear tip tubes 16 as shown in FIG. 5. Alternatively, arms 81 and 82 are elements of a system or apparatus other than a head piece.

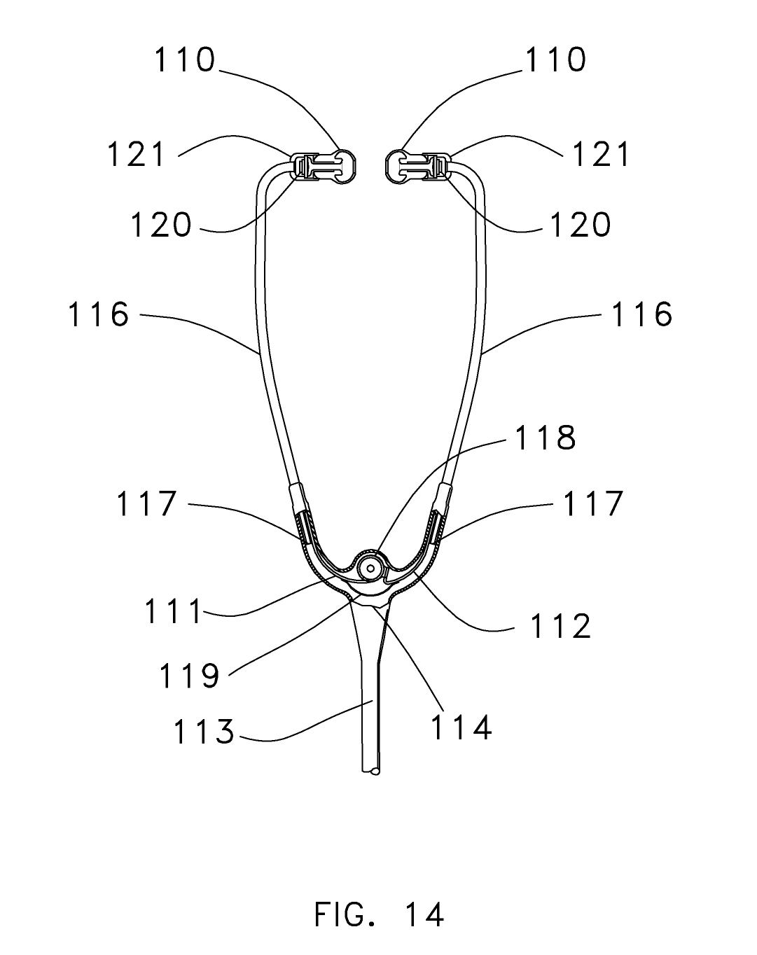

An optional addition to any embodiment of the inventive stethoscope (spring 119) is shown in FIG. 14. FIG. 14 shows an electronic stethoscope, but other embodiments of the inventive stethoscope can be implemented with a spring 119 or a variation on spring 119. The stethoscope of FIG. 14 includes a signal carrying tube 113, which has a chest piece (not shown) attached to its lower end and a “Y” section 114 at its upper end. The stethoscope of FIG. 14 also includes a head piece coupled to section 114. The head piece includes head piece tubes 116 (sometimes referred to herein as ear tip tubes 116), and an ear tip assembly 110, 120, and 121 at the end of each tube 116. Each ear tip assembly includes an end portion 110 (for placement in the meatus of a user's ear), a sound transducer housing 121 coupled to an end of tube 116, and a sound transducer 120 in the housing 121. In a typical electronic stethoscope implementation, tubes 116 would include necessary signal wires (not shown) extending between the stethoscope's electronics (which generate at least one electrical signal indicative of detected acoustic waves) and sound transducers 120 (which generate output sound audible to the user in response to the electrical signal or signals).

FIG. 14 shows a portion of tube 113 cut away to show an embodiment of the inventive coupling mechanism (111, 112, and 118) coupled between the lower ends of tubes 116, and the open lower ends (117) of tubes 116 inside the open upper ends of “Y” section 114 of tube 113. This transition between tube 113 and tubes 116 facilitates the passage of wires from the chest piece (not shown) to output sound transducers 120.

Arms 111 and 112 extend out from pivot mechanism 118 and are attached to the ear tip assembly tubes 116. Pulling outward on the two tubes 116 (i.e., pulling the left tube 116 leftward and the right tube 116 rightward in FIG. 14) causes pivot mechanism 118 (sometimes referred to herein as “pivot” 118) to allow counter-clockwise rotational movement of arm 111 and clockwise rotational movement of arm 112 with a predetermined first friction. Since arms 111 and 112 are attached to tubes 116, such pulling of tubes 116 allows the ear tip assemblies 110, 119, 120 to open outward from each other. When ear tip assemblies 110, 119, 120 are sufficiently separated from each other, the physician can then place the ear tip assemblies 110, 119, 120 into his ears. Movement of ear tip assemblies 110, 119, 120 toward each other (into the physician's ear canals) causes tubes 116 to reverse the rotation of pivot 118. The pivot 118 allows the latter movement with no friction (or no more than an insignificant amount of friction relative to the first predetermined friction) because it contains a one way roller clutch bearing 20 of the type described with reference to FIGS. 4a, 4b, and 5.

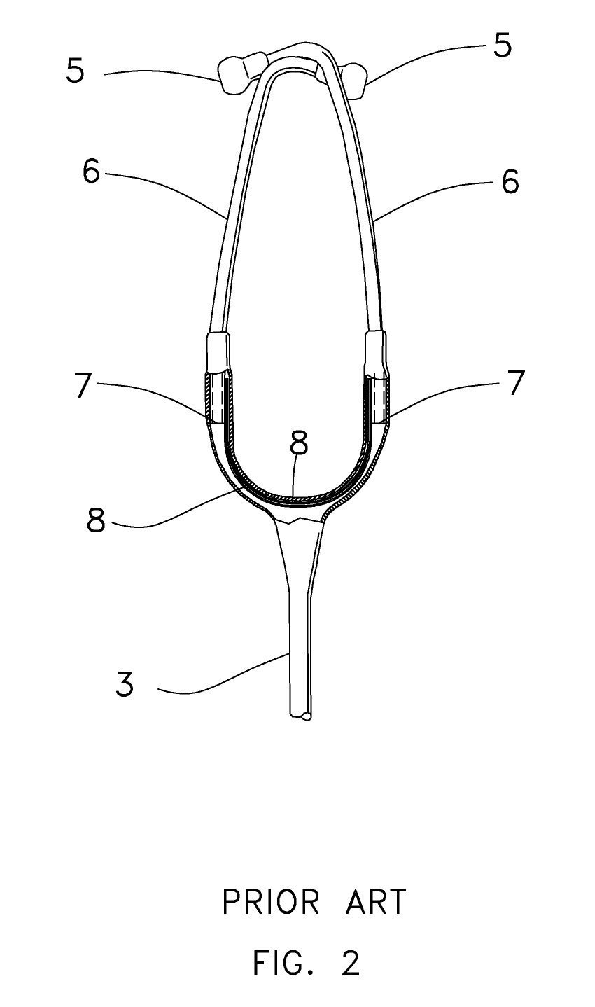

It has been found in some rare instances that a physician may desire some small bias in the order of about half an ounce on the ear tip. In such cases, a small leaf spring 119 can be provided between arms 111 and 112. The leaf spring can be manufactured to give a very gentle bias while still retaining the locking features of preferred embodiments of the invention. This is not possible in conventional prior art designs such as that illustrated in FIG. 2 since a very gentle spring would not support said stethoscope on the physician's ears, and would easily fall off.

It should be appreciated that if an embodiment of the inventive adjustment mechanism is included in an active stethoscope, amplified signals from an acoustic transducer (e.g., microphone) in the stethoscope's chest piece may be sent along wires past the inventive adjustment mechanism and through the stethoscope's ear tip tubes to output sound transducers (e.g., small loudspeakers) at the ear tips. More generally, transducer output signals (not necessarily electrical signals) may be sent from an acoustic transducer in the stethoscope's chest piece through or past the inventive adjustment mechanism (and then through the stethoscope's ear tip tubes to ear tips, or to processing circuitry and/or an additional transducer coupled acoustically, electronically, or by other means to ear tips).

In a class of embodiments, the invention is an asymmetric adjustment mechanism. In typical embodiments in this class, the mechanism is configured for use as a head piece adjustment mechanism in a stethoscope head piece or other head piece. For example, the asymmetric adjustment mechanism of FIG. 10 or FIG. 13 can be included in a head piece other than a stethoscope head piece. Alternatively, the inventive adjustment mechanism is used (or configured for use) in another system or apparatus for adjusting orientation of a first member relative to a second member (e.g., relative orientation of arms 81 and 82 of FIG. 13 in cases in which these arms are not elements of a head piece). Examples of such an adjustment mechanism (which is preferably a spring clutch device) include the asymmetric adjustment mechanism of FIG. 9, FIG. 10, or FIG. 13, and any of many variations thereon, including variations in which the adjustment mechanism is configured to be attached to members (whose relative orientation is to be adjusted) that are shaped differently than arms 41 and 42 of FIG. 10 or arms 81 and 82 of FIG. 13. Embodiments of the inventive adjustment mechanism are useful to adjust the position of a consumer appliance door (e.g., a springless oven door, a springless horizontal refrigerator or freezer door, or another refrigerator or freezer door), an automobile (or other motor vehicle) door, a non slamming house door or gate (e.g., a springless gate), or a laptop display screen. Herein, “laptop” denotes a laptop or notebook computer, tablet PC, PDA (personal digital assistant), or other portable computer or computing system. For example, a door, gate, or screen can be mounted to one or more of the inventive adjustment assemblies, with each adjustment assembly serving as a hinge. For example, a door, gate, or screen mounted on such a hinge (or hinges) can be opened by applying relatively small opening force thereto or closed by applying relatively large closing force thereto, and will remain locked in a user-determined orientation when the user ceases to apply opening or closing force thereto. In preferred embodiments, the asymmetric adjustment mechanism is a spring clutch device (e.g., identical or similar to that of FIG. 10 or FIG. 13).

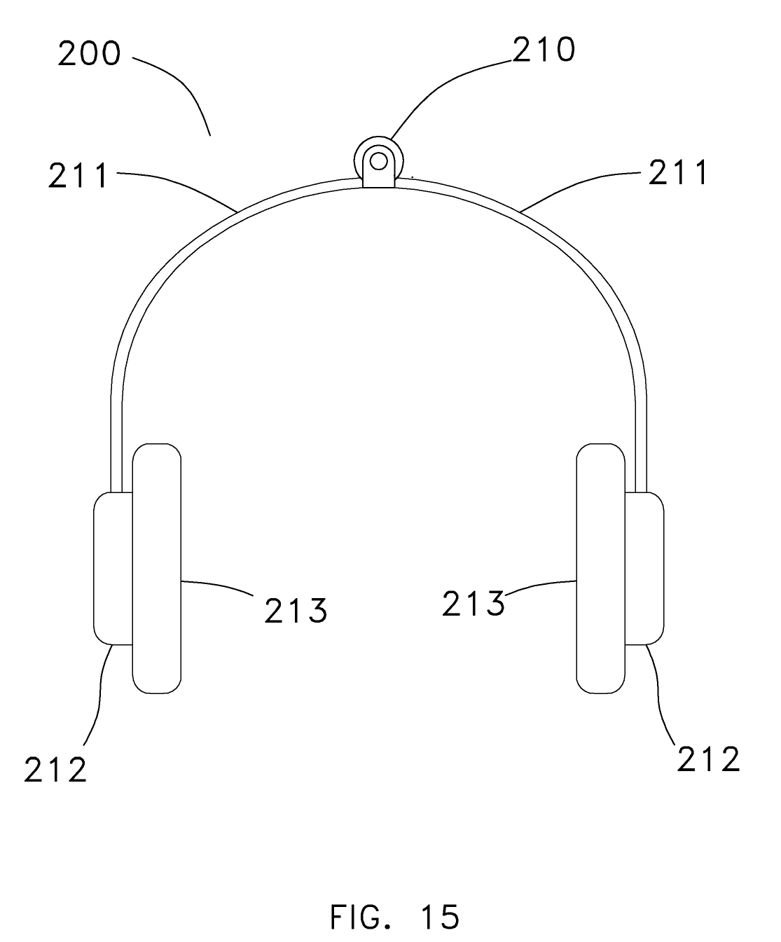

FIG. 15 shows a non-stethoscope application of the inventive coupling mechanism included in a headset 200. The FIG. 15 assembly includes an embodiment of the inventive adjustment mechanism 210, and left and right arms 211 coupled respectively between mechanism 210 and left and right output sound transducers 212. Suitable ear cushions 213 are attached to transducers 212. Adjustment of headset 200 is identical to adjustment of the stethoscope applications taught in this specification, except that arms 211 operate over the user's head instead of under the user's chin. Adjustment mechanism 210 can be any of many embodiments of the inventive adjustment mechanism, including any of those described herein. In typical embodiments, headset 200 can be closed (adjusted so as to reduce the separation between transducers 212) by exerting closing force (of magnitude in excess of a first threshold) on elements 211, 212, and/or 213 to move mechanism 210 by a desired amount, or can be opened (so as to increase separation between transducers 212) only by exerting opening force of magnitude in excess of a second threshold (greater than the first threshold) on elements 211, 212, and/or 213, and mechanism 210 enters a locked state whenever no opening force having magnitude in excess of the second threshold (or closing force having magnitude in excess of the first threshold) is exerted on elements 211, 212, and/or 213.

In typical embodiments of the inventive adjustment mechanism (e.g., typical spring clutch embodiments), the “high force” direction of rotational movement (the rotational direction which requires application of relatively high minimum force to move the adjustment mechanism) is adjustable (e.g., by appropriate implementation of a release spring of the mechanism and elements that interact with the release spring, and tangs of a locking spring of the mechanism). Preferably, asymmetric force required to rotate a first member of the adjustment mechanism relative to a second member thereof (or relative to an ear tip or other member coupled to the adjustment mechanism) is adjustable in both the clockwise direction and counterclockwise direction (i.e., in both the “high force” direction and “low force” direction of rotational movement). For example, in the spring clutch adjustment mechanism of FIG. 10, release spring 54, tangs 55 and 58, and pin 57 can have any of various different implementations and/or relative positions that require different amounts of threshold opening force for causing intermittent “opening” rotation of the spring clutch adjustment mechanism (to increase, intermittently, the separation between the free ends of arms 41 and 42). Also in the mechanism of FIG. 10, locking spring 51 can have any of various different implementations and/or can be wound more or less tightly around shaft 52 (e.g., during manufacture, shaft 52 can be sized appropriately relative to the inside diameter of spring 51, with larger diameter of shaft 52 giving higher friction in the “low force” direction and smaller shaft diameter giving lower friction in the “low force” direction) to require different amounts of threshold “closing” force for causing spring 51 to slip freely relatively to shaft 52 to decrease the separation between the free ends of arms 41 and 42.

Although it is typically preferable for the adjustment mechanism to offer independent adjustment capability in either rotational direction, in some embodiments of the adjustment mechanism, asymmetric force required to rotate a first member of the adjustment mechanism relative to a second member thereof (or relative to an ear tip or other member coupled to the adjustment mechanism) is adjustable (e.g., adjustable predominantly) in one rotational direction and minimally (or not at all) in the other rotational direction. For example, in the adjustment mechanism of FIG. 5, arm 11 can be implemented in various different ways (during manufacture) to require different amounts of threshold opening force for bending arm portion 29 of arm 11 sufficiently to allow bearing 20 to slip relative to the arm portion 29 (to increase the separation between the free ends of arms 11 and 14). However, the threshold “closing” force (for decreasing the separation between the free ends of arms 11 and 14 of the FIG. 5 embodiment) is not significantly adjustable.

In preferred embodiments, the inventive adjustment mechanism has a dominant characteristic of opening that is “non energy storing,” in the following sense. The “characteristic of opening” is a physical mechanism by which the mechanism can be opened (e.g., to increase separation between ear tips coupled thereto) in response to exertion of opening force thereon (typically only in response to exertion of opening force having magnitude in excess of a threshold magnitude thereon). The characteristic of opening is “non energy storing” in the sense that it doesn't rely upon conversion of potential energy stored in a spring, as of commencement of exertion of opening force on the mechanism, to open the mechanism in response to the opening force. For example, the adjustment mechanism of FIGS. 10-12 in its locked state has a dominant characteristic of opening in which clockwise rotation of arm 42 (in response to opening force having magnitude in excess of a threshold magnitude) advances one end of spring 54 against tang 55 of spring 51, causing instant lockup of spring 51 against shaft 52, followed by compression of spring 54 against tang 55 which allows additional rotation of hub 53 by a few degrees causing pin 57 to impinge on and push tang 58 to open spring 51 and defeat its lockup against shaft 52, followed by further compression of spring 54 against tang 55 which again closes spring 51 (causing instant lockup against shaft 52), and so on in a repeating cycle (to open the mechanism in stepwise fashion). When the adjustment mechanism of FIGS. 10-12 is in its locked state (e.g., when no opening or closing force is exerted thereon), springs 54 and 51 are relaxed and do not store potential energy that is relied upon to open the mechanism (i.e., if and when opening force having magnitude in excess of the threshold magnitude is exerted on the adjustment mechanism in the locked state). Thus, the adjustment mechanism of FIGS. 10-12 has a dominant characteristic of opening that is non energy storing.

In preferred embodiments, the inventive adjustment mechanism has a dominant characteristic of closing that is “non energy storing,” in the following sense. The “characteristic of closing” is a physical mechanism by which the mechanism can be closed (e.g., to decrease separation between ear tips coupled thereto) in response to exertion of closing force thereon (typically only in response to exertion of closing force having magnitude in excess of a threshold magnitude thereon). The characteristic of closing is “non energy storing” in the sense that it doesn't rely upon conversion of potential energy stored in a spring, as of commencement of exertion of closing force on the mechanism, to close the mechanism in response to the closing force. For example, the adjustment mechanism of FIGS. 10-12 in its locked state has a dominant characteristic of closing in which counterclockwise rotation of arm 42 (in response to closing force having magnitude in excess of a threshold magnitude) moves side wall 56a of hub 53 against tang 55 of spring 51, causing spring 51 to slip freely relatively to shaft 52, in turn allowing arm 42 to continue to rotate toward arm 41, closing the mechanism.). When the adjustment mechanism of FIGS. 10-12 is in its locked state (e.g., when no opening or closing force is exerted thereon), springs 54 and 51 are relaxed and do not store potential energy that is relied upon to open the mechanism (i.e., if and when closing force having magnitude in excess of the threshold magnitude is exerted on the adjustment mechanism in the locked state). Thus, the adjustment mechanism of FIGS. 10-12 has a dominant characteristic of closing that is non energy storing.

Although the descriptions above contain many specificities these should not be construed as limiting the scope of the invention but as merely providing illustrations of some of the presently preferred embodiments of this invention. The scope of the invention should be determined by the appended claims and their legal equivalents rather than by the examples provided.

What is claimed is:

1. A head piece, including:
an ear tip subassembly, including ear tips; and
an adjustment mechanism coupled to the ear tip subassembly and configured to enter a locked state in response to cessation of adjustment force exertion on the ear tip subassembly, wherein the adjustment mechanism in the locked state does not exert bias force on the ear tip subassembly having magnitude in excess of a threshold magnitude, and the adjustment mechanism is configured to close to reduce separation between the ear tips in response to closing force on the ear tip subassembly, and to open to increase separation between the ear tips only in response to opening force on the ear tip subassembly having magnitude greater than the threshold magnitude.

2. The head piece of claim 1, wherein the adjustment mechanism in the locked state exerts substantially no spring bias force on the ear tip subassembly.

3. The head piece of claim 1, wherein the adjustment mechanism in the locked state is configured to close, to reduce separation between the ear tips, in response to closing force on the ear tip subassembly having magnitude less than the threshold magnitude.

4. The head piece of claim 1, wherein said head piece is a stethoscope head piece.

5. The head piece of claim 1, wherein said head piece is an audio headset.

6. The head piece of claim 1, wherein the adjustment mechanism is a spring clutch device.

7. The head piece of claim 6, wherein the spring clutch device includes:
a shaft;
a locking spring around the shaft; and
a control assembly coupled to the ear tip subassembly, and movable in response to torque exerted thereon by the ear tip subassembly between positions in engagement with the locking spring, wherein the control assembly is configured to exert unlocking torque on the locking spring, to free the locking spring to rotate relative to the shaft, in response to closing torque exerted on the control assembly by the ear tip subassembly, and to exert locking torque on the locking spring in response to opening torque exerted on the control assembly by the ear tip subassembly.

8. The head piece of claim 7, wherein the spring clutch device is configured to respond to opening torque of magnitude greater than a threshold value on the control assembly by sequentially locking and then unlocking the locking spring relative to the shaft.

9. The head piece of claim 8, wherein the ear tip subassembly includes ear tip supports that support the ear tips, and the control assembly includes:
a first subassembly coupled to one of the ear tip supports; and
a second subassembly coupled to another one of the ear tip supports so as to be rotatable relative to the first subassembly when the spring clutch device is not in the locked state, wherein at least one of the first subassembly and the second subassembly is coupled to the shaft, and wherein movement of the spring clutch device in response to said opening torque of magnitude greater than the threshold value repeatedly locks and then unlocks the locking spring relative to the shaft, allowing intermittent rotation of the second subassembly relative to the first subassembly.

10. The head piece of claim 7, wherein the ear tip subassembly includes ear tip supports that support the ear tips, and the control assembly includes:
a first assembly coupled to one of the ear tip supports;
a second assembly, coupled to another one of the ear tip supports so as to be rotatable relative to the first assembly when the adjustment mechanism is not in the locked state, wherein at least one of the first assembly and the second assembly is coupled to the shaft; and
a release spring between the locking spring and at least one of the first assembly and the second assembly, wherein the control assembly is configured to respond to opening torque of magnitude greater than a threshold value exerted thereon by the ear tip subassembly by repeatedly locking and then unlocking the locking spring relative to the shaft, and wherein movement of the release spring in response to force exerted thereon by at least one of the first assembly and the second assembly in response to said opening torque triggers each said unlocking of the locking spring.

11. The head piece of claim 10, wherein the first assembly is a hub assembly that defines a spring-retaining cavity, and the release spring is positioned in the spring-retaining cavity.

12. The head piece of claim 10, wherein the release spring is a sheet spring having two openings extending therethrough, the first assembly is attached to the sheet spring, the sheet spring is positioned so as to partially surround the locking spring with portions of the locking spring protruding into the sheet spring's openings, whereby movement of the sheet spring in response to force exerted thereon by the first assembly in response to the opening torque repeatedly locks and then unlocks the locking spring relative to the shaft, allowing intermittent rotation of the second assembly relative to the first assembly.

13. The head piece of claim 12, wherein the sheet spring has a generally split cylindrical shape.

14. The head piece of claim 13, wherein a first one of the openings through the sheet spring is elongated, and the other of the openings through the sheet spring is less elongated than the first one of the openings.

15. The head piece of claim 13, wherein the sheet spring is made of spring steel sheet stock.

16. The head piece of claim 7, wherein the spring clutch device in the locked state is configured to close, to reduce separation between the ear tips, in response to closing force on the ear tip subassembly having magnitude less than the threshold magnitude, but exertion of opening force on the ear tip subassembly having magnitude greater than the threshold magnitude is required to open the spring clutch device in said locked state to increase separation between the ear tips.

17. The head piece of claim 1, wherein the adjustment mechanism includes a roller clutch bearing.

18. The head piece of claim 17, wherein the ear tip subassembly includes ear tip supports that support the ear tips, and the adjustment mechanism includes:
a first assembly attached to one of the ear tip supports; and
a second assembly attached to a second one of the ear tip supports, wherein the second assembly includes the roller clutch bearing, the first assembly includes a shaft surrounded at least partially by the roller clutch bearing with freedom to rotate relative to said roller clutch bearing when the adjustment mechanism is not in the locked state, and the roller clutch bearing is locked relative to the shaft when the adjustment mechanism is in the locked state.

19. The head piece of claim 18, wherein the first assembly is fixedly attached to said one of the ear tip supports, and the second assembly is coupled to the second one of the ear tip supports so as not to slip relative to the roller clutch bearing in response to opening force on the ear tip subassembly having magnitude less than the threshold magnitude but to slip relative to the roller clutch bearing in response to opening force on the ear tip subassembly having magnitude greater than the threshold magnitude.

20. A spring clutch device for adjusting orientation of a first member relative to a second member, said spring clutch device including:
a shaft;
a locking spring around the shaft; and
a control assembly configured to be coupled to the first member and to the second member, and to be movable between positions in engagement with the locking spring in response to torque exerted thereon by at least one of the first member and the second member when said first member and said second member are coupled thereto, wherein the control assembly is configured to exert unlocking torque on the locking spring to free the locking spring to rotate relative to the shaft, in response to closing torque exerted on the control assembly by at least one of the first member and the second member when the first member and the second member are coupled to the control assembly, and to exert locking torque on the locking spring in response to opening torque exerted on the control assembly by at least one of the first member and the second member when said first member and said second member are coupled to the control assembly.

21. The device of claim 20, wherein said device is configured to enter a locked state in response to cessation of adjustment force exerted thereon by the first member and the second member when said first member and said second member are coupled to the control assembly, and wherein the device in the locked state with the first member and the second member coupled to the control assembly does not exert bias force on the first member in excess of a threshold magnitude and does not exert bias force on the second member in excess of the threshold magnitude, and the control assembly with the first member and the second member coupled thereto is configured to close to rotate the first member toward the second member in response to closing force on said first member and said second member, and to open to rotate said first member away from said second member only in response to opening force on said first member and said second member having magnitude greater than the threshold magnitude.

22. The device of claim 21, wherein said device is configured to exert substantially no spring bias force on the first member and the second member, when said device is in the locked state with said first member and said second member coupled to the control assembly.

23. The device of claim 20, wherein said device is configured to respond to opening torque of magnitude greater than a threshold value on the control assembly by sequentially locking and then unlocking the locking spring relative to the shaft.

24. The device of claim 23, wherein said device is configured to enter a locked state in response to cessation of adjustment force exerted thereon by the first member and the second member when said first member and said second member are coupled to the control assembly, and wherein the control assembly includes:
a first subassembly configured to be coupled to the first member; and
a second subassembly configured to be coupled to the second member so as to be rotatable relative to the first subassembly when said device is not in the locked state, wherein at least one of the first subassembly and the second subassembly is coupled to the shaft, and wherein movement of said device in response to said opening torque of magnitude greater than the threshold value repeatedly locks and then unlocks the locking spring relative to the shaft, allowing intermittent rotation of the second subassembly relative to the first subassembly.

25. The device of claim 20, wherein said device is configured to enter a locked state in response to cessation of adjustment force exerted thereon by the first member and the second member when said first member and said second member are coupled to the control assembly, and wherein the control assembly includes:
a first assembly configured to be coupled to the first member;
a second assembly configured to be coupled to the second member so as to be rotatable relative to the first assembly when said device is not in the locked state, wherein at least one of the first assembly and the second assembly is coupled to the shaft; and
a release spring between the locking spring and at least one of the first assembly and the second assembly, wherein the control assembly is configured to respond to opening torque of magnitude greater than a threshold value thereon by repeatedly locking and then unlocking the locking spring relative to the shaft, with movement of the release spring in response to force exerted thereon by at least one of the first assembly and the second assembly in response to said opening torque triggering each said unlocking of the locking spring.

26. The device of claim 25, wherein the first assembly is a hub assembly that defines a spring-retaining cavity, and the release spring is mounted in the spring-retaining cavity.

27. The device of claim 25, wherein the release spring is a sheet spring having two openings extending therethrough, the first assembly is attached to the sheet spring, the sheet spring is positioned so as to partially surround the locking spring with portions of the locking spring protruding into the sheet spring's openings, whereby movement of the sheet spring in response to force exerted thereon by the first assembly in response to the opening torque repeatedly locks and then unlocks the locking spring relative to the shaft, allowing intermittent rotation of the second assembly relative to the first assembly.

28. The device of claim 27, wherein the sheet spring has a generally split cylindrical shape.

29. The device of claim 28, wherein a first one of the openings through the sheet spring is elongated, and the other of the openings through the sheet spring is less elongated than the first one of the openings.

30. The device of claim 28, wherein the sheet spring is made of spring steel sheet stock.

31. A stethoscope, including:
a chest piece; and
a head piece coupled to the chest piece, wherein the head piece includes:
an ear tip subassembly, including ear tips; and
an adjustment mechanism coupled to the ear tip subassembly and configured to enter a locked state in response to cessation of adjustment force exertion on the ear tip subassembly, wherein the adjustment mechanism in the locked state does not exert bias force on the ear tip subassembly having magnitude in excess of a threshold magnitude, and the adjustment mechanism is configured to close to reduce separation between the ear tips in response to closing force on the ear tip subassembly, and to open to increase separation between the ear tips only in response to opening force on the ear tip subassembly having magnitude greater than the threshold magnitude.

32. The stethoscope of claim 31, wherein the adjustment mechanism in the locked state exerts substantially no spring bias force on the ear tip subassembly.

33. The stethoscope of claim 31, wherein the adjustment mechanism in the locked state is configured to close, to reduce separation between the ear tips, in response to closing force on the ear tip subassembly having magnitude less than the threshold magnitude.

34. The stethoscope of claim 31, wherein the adjustment mechanism is a spring clutch device.

35. The stethoscope of claim 34, wherein the spring clutch device includes:
a shaft;
a locking spring around the shaft; and
a control assembly coupled to the ear tip subassembly, and movable in response to torque exerted thereon by the ear tip subassembly between positions in engagement with the locking spring, wherein the control assembly is configured to exert unlocking torque on the locking spring, to free the locking spring to rotate relative to the shaft, in response to closing torque exerted on the control assembly by the ear tip subassembly, and to exert locking torque on the locking spring in response to opening torque exerted on the control assembly by the ear tip subassembly.

36. The stethoscope of claim 35, wherein the spring clutch device is configured to respond to opening torque of magnitude greater than a threshold value on the control assembly by sequentially locking and then unlocking the locking spring relative to the shaft.

37. The stethoscope of claim 36, wherein the ear tip subassembly includes ear tip supports that support the ear tips, and the control assembly includes:
a first subassembly coupled to one of the ear tip supports; and
a second subassembly coupled to another one of the ear tip supports so as to be rotatable relative to the first subassembly when the spring clutch device is not in the locked state, wherein at least one of the first subassembly and the second subassembly is coupled to the shaft, and wherein movement of the spring clutch device in response to said opening torque of magnitude greater than the threshold value repeatedly locks and then unlocks the locking spring relative to the shaft, allowing intermittent rotation of the second subassembly relative to the first subassembly.

38. The stethoscope of claim 35, wherein the ear tip subassembly includes ear tip supports that support the ear tips, and the control assembly includes:
a first assembly coupled to one of the ear tip supports;
a second assembly, coupled to another one of the ear tip supports so as to be rotatable relative to the first assembly when the adjustment mechanism is not in the locked state, wherein at least one of the first assembly and the second assembly is coupled to the shaft; and
a release spring between the locking spring and at least one of the first assembly and the second assembly, wherein the control assembly is configured to respond to opening torque of magnitude greater than a threshold value exerted thereon by the ear tip subassembly by repeatedly locking and then unlocking the locking spring relative to the shaft, and wherein movement of the release spring in response to force exerted thereon by at least one of the first assembly and the second assembly in response to said opening torque triggers each said unlocking of the locking spring.

39. The stethoscope of claim 38, wherein the first assembly is a hub assembly that defines a spring-retaining cavity, and the release spring is positioned in the spring-retaining cavity.

40. The stethoscope of claim 38, wherein the release spring is a sheet spring having two openings extending therethrough, the first assembly is attached to the sheet spring, the sheet spring is positioned so as to partially surround the locking spring with portions of the locking spring protruding into the sheet spring's openings, whereby movement of the sheet spring in response to force exerted thereon by the first assembly in response to the opening torque repeatedly locks and then unlocks the locking spring relative to the shaft, allowing intermittent rotation of the second assembly relative to the first assembly.

41. The stethoscope of claim 39, wherein the sheet spring has a generally split cylindrical shape.

42. The stethoscope of claim 41, wherein a first one of the openings through the sheet spring is elongated, and the other of the openings through the sheet spring is less elongated than the first one of the openings.

43. The stethoscope of claim 35, wherein the spring clutch device in the locked state is configured to close, to reduce separation between the ear tips, in response to closing force on the ear tip subassembly having magnitude less than the threshold magnitude, but exertion of opening force on the ear tip subassembly having magnitude greater than the threshold magnitude is required to open the spring clutch device in said locked state to increase separation between the ear tips.

44. The stethoscope of claim 31, wherein the adjustment mechanism includes a roller clutch bearing.

45. The stethoscope of claim 44, wherein the ear tip subassembly includes ear tip supports that support the ear tips, and the adjustment mechanism includes:
a first assembly attached to one of the ear tip supports; and
a second assembly attached to another one of the ear tip supports, wherein the second assembly includes the roller clutch bearing, the first assembly includes a shaft surrounded at least partially by the roller clutch bearing with freedom to rotate relative to said roller clutch bearing when the adjustment mechanism is not in the locked state, and the roller clutch bearing is locked relative to the shaft when the adjustment mechanism is in the locked state.

46. The stethoscope of claim 31, wherein said stethoscope is an active stethoscope, and the chest piece includes:
a diaphragm; and
an acoustic transducer mounted to sense movement of the diaphragm.

47. An assembly, including:
a member; and
an adjustment mechanism coupled to the member and configured as a hinge that allows clockwise rotation of said member relative to the adjustment mechanism in response to closing force, on said member and said adjustment mechanism, having magnitude in excess of a threshold magnitude but not in response to closing force of magnitude less than the threshold magnitude, and allows counterclockwise rotation of said member relative to the adjustment mechanism in response to opening force on said member and said adjustment mechanism having magnitude in excess of a second threshold magnitude but not in response to opening force of magnitude less than the second threshold magnitude, where the second threshold magnitude is different than the threshold magnitude.

48. The assembly of claim 47, also including:
a second member coupled to the adjustment mechanism such that the second member has freedom to rotate clockwise relative to the member in response to closing force, on said member and said second member, of magnitude in excess of the threshold magnitude but not in response to closing force of magnitude less than the threshold magnitude, and to rotate counterclockwise relative to the member in response to opening force, on said member and said second member, of magnitude in excess of the second threshold magnitude but not in response to opening force of magnitude less than the second threshold magnitude.

49. The assembly of claim 48, wherein the hinge assembly is configured for use in a head piece including an ear tip subassembly, with the first member functioning as an ear tip of the ear tip subassembly and the second member as a second ear tip of the ear tip subassembly.

50. The assembly of claim 49, wherein at least one of the threshold magnitude and the second threshold magnitude is adjustable during at least one of manufacture, configuration, and use of the adjustment mechanism.

51. The assembly of claim 50, wherein at least one of the threshold magnitude and the second threshold magnitude is adjustable during manufacture of the adjustment mechanism.

52. The assembly of claim 49, wherein both the threshold magnitude and the second threshold magnitude are adjustable during at least one of manufacture, configuration, and use of the adjustment mechanism.

53. The assembly of claim 48, wherein the adjustment mechanism is a slip clutch.

54. The assembly of claim 48, wherein the adjustment mechanism is a spring clutch device.

55. The assembly of claim 54, wherein the spring clutch device includes:
a shaft;
a locking spring around the shaft; and
a control assembly coupled to the member and to the second member, and movable between positions in engagement with the locking spring in response to torque exerted thereon by at least one of the member and the second member, wherein the control assembly is configured to exert unlocking torque on the locking spring to free the locking spring to rotate relative to the shaft, in response to closing torque exerted on the control assembly by at least one of the member and the second member, and to exert locking torque on the locking spring in response to opening torque exerted on the control assembly by at least one of the member and the second member.

56. The assembly of claim 48, wherein the adjustment mechanism includes a roller clutch bearing.

57. The assembly of claim 56 wherein the adjustment mechanism includes:
a first assembly fixedly attached to the member; and
a second assembly including the roller clutch bearing, wherein the first assembly includes a shaft having freedom to rotate relative to said roller clutch bearing when the adjustment mechanism is not in a locked state, the roller clutch bearing is locked relative to the shaft when the adjustment mechanism is in the locked state, the second assembly is coupled to the second member so as not to slip relative to the roller clutch bearing in response to opening force on the member and the second member having magnitude less than the second threshold magnitude but to slip relative to the roller clutch bearing in response to opening force on the member and the second member having magnitude greater than the second threshold magnitude.

58. The assembly of claim 47, wherein at least one of the threshold magnitude and the second threshold magnitude is adjustable during at least one of manufacture, configuration, and use of the adjustment mechanism.

59. The assembly of claim 58, wherein at least one of the threshold magnitude and the second threshold magnitude is adjustable during manufacture of the adjustment mechanism.

60. The assembly of claim 47, wherein both the threshold magnitude and the second threshold magnitude are adjustable during at least one of manufacture, configuration, and use of the adjustment mechanism.

61. The assembly of claim 47, wherein the hinge assembly is configured for use in a head piece including an ear tip subassembly, with the member functioning as an ear tip of the ear tip subassembly.

62. The assembly of claim 47, wherein the adjustment mechanism is a slip clutch.

63. An assembly, including:
a member; and
an adjustment mechanism coupled to the member, wherein the assembly is configured as a hinge such that the member is rotatable clockwise and counterclockwise relative to the adjustment mechanism, at least a minimum force must be exerted on the adjustment mechanism to rotate the member clockwise relative to the adjustment mechanism, and the minimum force is asymmetric with respect to a second minimum force required to be exerted on the adjustment mechanism to rotate the member counterclockwise relative to said adjustment mechanism.

64. The assembly of claim 63, also including:
a second member coupled to the adjustment mechanism such that the hinge allows clockwise rotation of the member relative to the second member in response to opening force of magnitude in excess of a threshold magnitude on the member and the second member but not in response to opening force of magnitude less than the threshold magnitude on the member and the second member, and allows counterclockwise rotation of said member relative to said second member in response to closing force of magnitude in excess of a second threshold magnitude on said member and said second member but not in response to closing force of magnitude less than the second threshold magnitude on said member and said second member.

65. The assembly of claim 64, wherein at least one of the threshold magnitude and the second threshold magnitude is adjustable during at least one of manufacture, configuration, and use of said assembly.

66. The assembly of claim 64, wherein the threshold magnitude and the second threshold magnitude are independently adjustable during at least one of manufacture, configuration, and use of said assembly.

67. The assembly of claim 63, wherein at least one of the minimum force and the second minimum force is adjustable during at least one of manufacture, configuration, and use of said assembly.

68. The assembly of claim 63, wherein the minimum force and the second minimum force are independently adjustable during at least one of manufacture, configuration, and use of said assembly.

69. The assembly of claim 63, wherein the adjustment mechanism is a slip clutch.

70. The assembly of claim 63, wherein the adjustment mechanism is a spring clutch device.

71. The assembly of claim 63, wherein the adjustment mechanism includes a roller clutch bearing.

72. An adjustment mechanism, including:
a first assembly; and
a second assembly coupled to the first assembly such that the first assembly is rotatable clockwise and counterclockwise relative to the second assembly, wherein the adjustment mechanism is configured such that at least a minimum force must be exerted on the adjustment mechanism to rotate the first assembly clockwise relative to the second assembly, and the minimum force is asymmetric with respect to a second minimum force required to be exerted on the adjustment mechanism to rotate the first assembly counterclockwise relative to the second assembly.

73. The adjustment mechanism of claim 72, wherein the first assembly includes a member, and the second assembly includes a second member and a spring clutch device coupled to the member and to the second member.

74. The adjustment mechanism of claim 73, wherein the spring clutch device includes:
a shaft;
a locking spring around the shaft; and
a control assembly coupled to the member and to the second member, and movable between positions in engagement with the locking spring in response to torque exerted thereon by at least one of the member and the second member, wherein the control assembly is configured to exert unlocking torque on the locking spring to free the locking spring to rotate relative to the shaft, in response to closing torque exerted on the control assembly by at least one of the member and the second member, and to exert locking torque on the locking spring in response to opening torque exerted on the control assembly by at least one of the member and the second member.

75. The adjustment mechanism of claim 72, wherein the second assembly includes a member and a roller clutch bearing coupled to the member and to the first assembly.

76. The adjustment mechanism of claim 75, wherein said adjustment mechanism has a locked state, the first assembly includes a shaft having freedom to rotate relative to the roller clutch bearing when the adjustment mechanism is not in the locked state, the roller clutch bearing is locked relative to the shaft when the adjustment mechanism is in the locked state, and the member is coupled to roller clutch bearing so as not to slip relative to said roller clutch bearing in response to opening force on the member and the first assembly having magnitude less than a threshold magnitude but to slip relative to the roller clutch bearing in response to opening force on the member and the first assembly having magnitude greater than the threshold magnitude.

77. The adjustment mechanism of claim 72, wherein a cylindrical clutch bearing is included in at least one of the first assembly and the second assembly.

78. A head piece, including:
an ear tip subassembly, including ear tips; and
an adjustment mechanism coupled to the ear tip subassembly and configured to close to reduce separation between the ear tips in response to closing force on the ear tip subassembly having magnitude greater than a threshold magnitude but not in response to closing force having magnitude less than the threshold magnitude, and to open to increase separation between the ear tips in response to opening force on the ear tip subassembly having magnitude greater than a second threshold magnitude but not in response to opening force having magnitude less than the second threshold magnitude, where the second threshold magnitude is different than the threshold magnitude.

79. The head piece of claim 78, wherein the adjustment mechanism whose dominant characteristic of opening is non energy storing.

80. The head piece of claim 79, wherein the adjustment mechanism whose dominant characteristic of closing is non energy storing.

81. The head piece of claim 78, wherein the adjustment mechanism whose dominant characteristic of closing is non energy storing.

82. The head piece of claim 78, wherein the head piece is included in a stethoscope, and the stethoscope also includes:
a chest piece coupled to the head piece.

83. The head piece of claim 82, wherein the stethoscope is an active stethoscope, the chest piece includes a diaphragm and an acoustic transducer mounted to sense movement of the diaphragm, and the ear tip subassembly includes electrical to sound transducers coupled to the acoustic transducer and mounted in the ear tips for generating acoustic waves in response to output of the acoustic transducer.

84. The head piece of claim 78, wherein the ear tip subassembly includes electrical to sound transducers in the ear tips.
